(19) United States
(12) Patent Application Publication
(10) Pub. No.: US 2021/0130408 A1
(43) Pub. Date: May 6, 2021

(54) COMPOUND AND METHOD FOR PRODUCING THE SAME

(71) Applicant: PUSAN NATIONAL UNIVERSITY INDUSTRY-UNIVERSITY COOPERATION FOUNDATION, Busan (KR)

(72) Inventors: Il KIM, Busan (KR); Hua JIN, Busan (KR); Yu ZHANG, Busan (KR)

(21) Appl. No.: 16/633,701

(22) PCT Filed: Jul. 24, 2018
(86) PCT No.: PCT/KR2018/008328

§ 371 Date: Jan. 24, 2020

(30) Foreign Application Priority Data
Jul. 24, 2017 (KR) 10-2017-0093530

(51) Int. Cl. C07K 7/50; C07K 14/00; C08G 69/10

(52) U.S. Cl. CPC C07K 7/50; C08G 69/10; C07K 14/001

(57) ABSTRACT

Disclosed is a method for producing a compound, the method including polymerizing an amino acid carboxyanhydride-based compound using a catalyst. The method for producing the compound may improve a polymerization reaction rate and provide a compound having a narrower molecular weight distribution and having a polymer ring structure bonded to the catalyst.

### TECHNICAL FIELD

The present disclosure relates to a compound and a method for producing the same. More specifically, the present disclosure relates to a compound that may be used for various purposes in a field of biotechnology and to a method for producing the same.

### BACKGROUND ART

A hybrid material based on a polypeptide as a compound with a cyclic or linear structure includes characteristics of sequence order control, functional control, a regular form (e.g., a spiral form, a folding screen form, a corner form), special stereochemistry, biocompatibility, and biodegradability. Because of those characteristics, the hybrid material based on the polypeptide has received a lot of attention. The polypeptide based compound is widely used in nanobiotechnology such as drug delivery, artificial tissues and transplantation, biological mineral generation, medical diagnostics, and colloidal chemical analysis using biosensors.

A cyclic peptide has received a lot of attention because the cyclic peptide has unique features due to a limited steric conformation compared to a linear peptide. These features include a new colloidal form, a faster crystallization rate, a lower intrinsic viscosity, and a higher glass transition temperature and a higher melting point. Further, the cyclic peptide has a characteristic of a building block for self-assembly and allows formation of a self-assembled peptide nanostructure having a stable secondary structure, abnormally high thermal stability, and well controlled morphological characteristics.

Various methods for producing the peptide have been developed so far, but all of the methods have advantages and disadvantages. For example, a metal catalyst used in synthesis of the peptide causes nonspecific toxicity and must be completely removed when the compound is used as a biomaterial. The synthesis of the peptide using the metal catalyst requires high vacuum technology requiring complex and expensive laboratory equipment. Further, trifluoroborane and silazane used as an initiator are sensitive to a hydration reaction. In addition, a cumbersome synthesis process is required and a long reaction duration of more than about 48 hours make the synthesis impractical.

A ring-opening polymerization of α-amino acid N-carboxyanhydride free of the metal catalyst related problems and a by-product is most commonly used for producing the peptide. However, in this peptide synthesis method, two mechanisms, that is, a normal amine mechanism (NMR) and an activated monomer mechanism (AMM) coexist. Because these two mechanisms compete with each other to affect a polymerization process, a molecular weight may not be controlled and a molecular weight distribution is broad. In addition, because the synthesis consumes more than three days, a method for controlling the molecular weight distribution and reducing the synthesis time is required. Although peptide cyclization methods have been developed conventionally, most thereof are related to a cyclization reaction of a low molecular weight peptide having a number of amino acids smaller than 20. Therefore, there is a need for a method for producing a large cyclic peptide in a faster manner and at a higher yield.

### DISCLOSURE

### Technical Purposes

One purpose of the present disclosure is to provide a cyclic compound and a method for producing the same.

Another purpose of the present disclosure is to provide a method for producing a compound in which a synthesis duration thereof is shortened and a high yield is achieved and a molecular weight distribution is controlled.

### Technical Solutions

A compound for achieving one purpose of the present disclosure is represented by a following Chemical Formula 1:

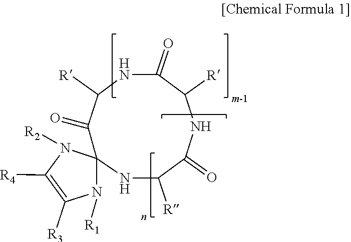

In the Chemical Formula 1, each of R₁ to R₄ independently represents a hydrogen atom, an alkyl group having 1 to 20 carbon atoms, a cycloalkyl group having 3 to 20 carbon atoms, an ethylene glycol group having 3 to 50 carbon atoms, an aryl group having 6 to 20 carbon atoms, or a cycloalkenyl group having 5 to 20 carbon atoms.

Each of R′ and R″ independently represents R-A-(CH₂)ₓ—*, where A represents a single bond, a sulfur atom (—S—), an oxygen atom (—O—), a nitrogen atom (—N—),

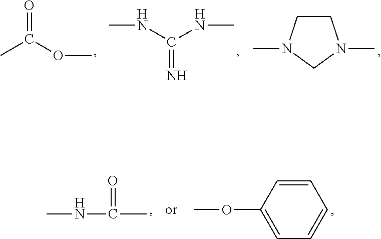

and R represents a hydrogen atom, a halogen atom, an alkyl group having 1 to 5 carbon atoms, an aryl group having 6 to 15 carbon atoms, a carbobenzoxy group, a trifluoroacetyl group, a carbonyl group, a triphenylmethyl group, a methoxydiphenylmethyl group, a 2,4,6-trimethoxybenzyl group, or a 2,2,4,6,7-pentamethyldihydrobenzofuran-5-sulfonyl group, where x represents an integer of 0 or greater.

A hydrogen atom of each of R₁ to R₄, R′ and R″ may be independently substituted or unsubstituted with a substituent selected from a group consisting of a halogen atom, a sulfur atom, an oxygen atom, a hydroxy group, an amine group, an ether group, a carbonyl group, an alkenyl group, an allyl group, a phenyl group, and a cyano group, where n is an integer greater than or equal to 0, and m is an integer of 1 or greater.

In one embodiment, each of R₁ and R₂ may independently represent a cycloalkyl group, an alkyl group or an aryl group unsubstituted or substituted with the substituent.

In one embodiment, each of R₁ and R₂ may independently represent an alkyl group having 1 to 20 carbon atoms or a cycloalkyl group having 6 to 20 carbon atoms, and each of R₃ and R₄ may independently represent a hydrogen atom.

A method for producing a compound to achieve another purpose of the present disclosure includes polymerizing α-amino acid N-carboxyanhydride using a catalyst represented by a following Chemical Formula 2:

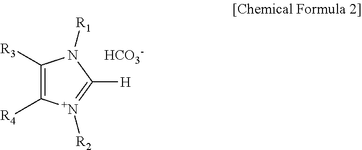

In the Chemical Formula 2, each of R₁ to R₄ independently represents a hydrogen atom, an alkyl group having 1 to 20 carbon atoms, a cycloalkyl group having 3 to 20 carbon atoms, ethylene glycol having 3 to 50 carbon atoms, an aryl group having 6 to 20 carbon atoms or a cycloalkenyl group having 5 to 20 carbon atoms.

A hydrogen atom of each of the alkyl group, the cycloalkyl group, the aryl group and the cycloalkenyl group may be independently substituted or unsubstituted with a substituent selected from a group consisting of an ether group, a carbonyl group, an alkenyl group, an allyl group, a halogen atom, a hydroxy group, a phenyl group, and a cyano group.

In one embodiment, each of R₁ and R₂ in the catalyst represented by the Chemical Formula 2 may independently represent a cycloalkyl group, an alkyl group or an aryl group unsubstituted or substituted with the substituent.

In one embodiment, the catalyst represented by the Chemical Formula 2 may include at least one of compounds represented by following Chemical Formulas 2-1, 2-2, 2-3 and 2-4.

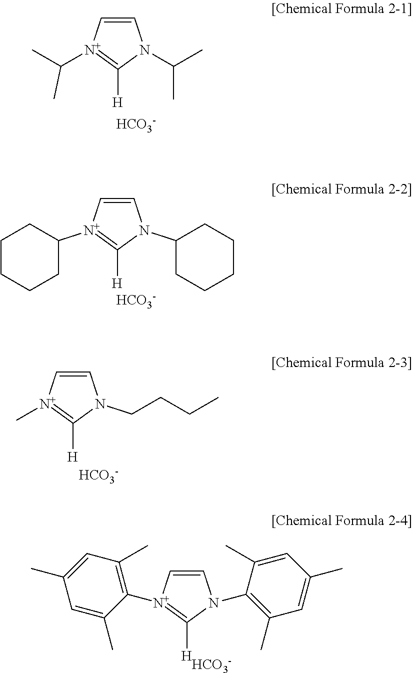

In one embodiment, the α-amino acid N-carboxyanhydride may be represented by a following Chemical Formula 3:

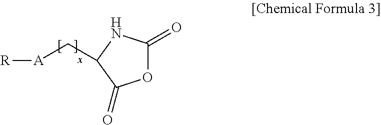

In the Chemical Formula 3, A represents a single bond, a hydrogen atom (—H—), a sulfur atom (—S—), an oxygen atom (—O—), a nitrogen atom (—N—),

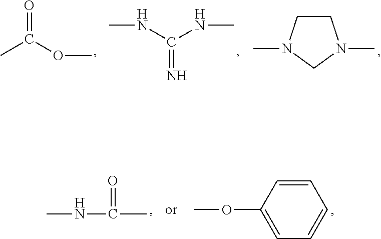

and R represents a hydrogen atom, a halogen atom, an alkyl group having 1 to 5 carbon atoms, an aryl group having 6 to 15 carbon atoms, a carbobenzoxy group, a trifluoroacetyl group, a carbonyl group, a triphenylmethyl group, a methoxydiphenylmethyl group, a 2,4,6-trimethoxybenzyl group, or a 2,2,4,6,7-pentamethyldihydrobenzofuran-5-sulfonyl group, where x represents an integer of 0 or greater.

In one embodiment, the α-amino acid N-carboxyanhydride may include at least one selected from a group consisting of protected or unprotected L-glycine N-carboxyanhydride, L-alanine N-carboxyanhydride, L-phenylalanine N-carboxyanhydride, L-valine N-carboxyanhydride, L-luecine N-carboxyanhydride, L-methlonine N-carboxyanhydride, L-isoleucine N-carboxyanhydride, L-proline N-carboxyanhydride, L-tryptophan N-carboxyanhydride, L-serine N-carboxyanhydride, L-cysteine N-carboxyanhydride, L-aspartic acid N-carboxyanhydride, L-glutamate N-carboxyanhydride, L-lysine N-carboxyanhydride, L-arginine N-carboxyanhydride, L-histidine N-carboxyanhydride, L-asparagine N-carboxyanhydride, L-glutamine N-carboxyanhydride, L-threonine N-carboxyanhydride, and L-tyrosine N-carboxyanhydride.

In one embodiment, the α-amino acid N-carboxyanhydride may include at least one of compounds represented by following Chemical Formulas A, B, C, D, E, F, G, H, I, J and K.

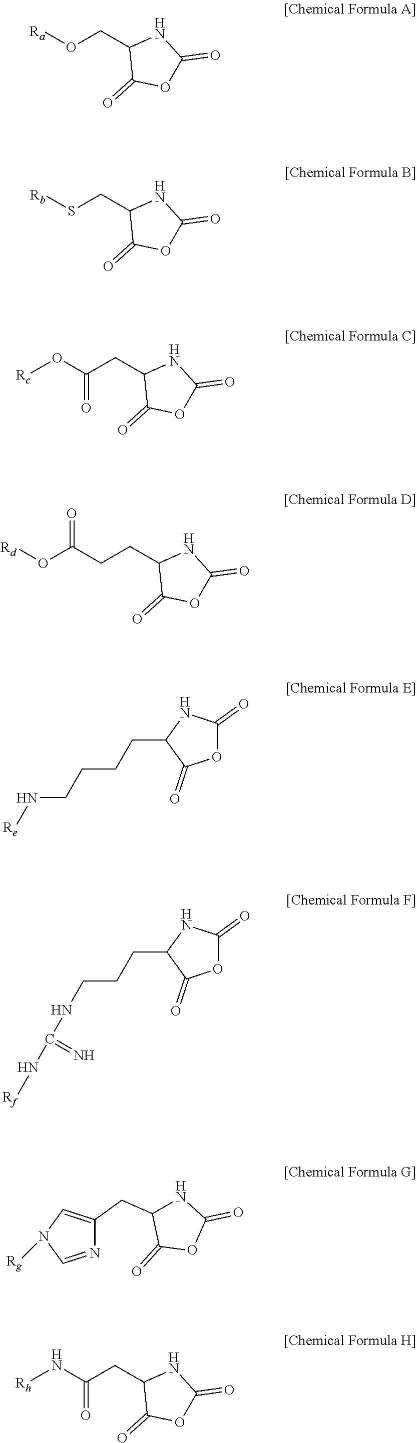

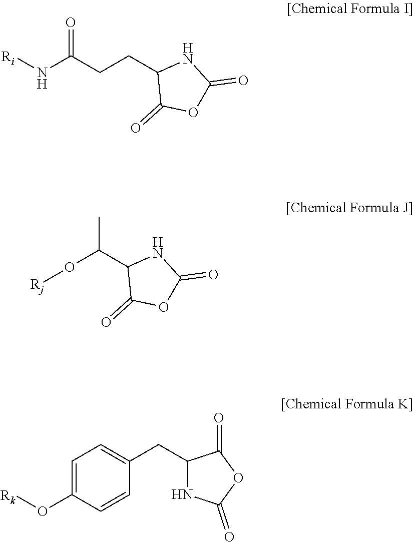

Each of Rₐ to Rₖ independently represents a hydrogen atom, a halogen atom, an alkyl group having 1 to 5 carbon atoms, an aryl group having 6 to 15 carbon atoms, a carbonyl group, a carbobenzoxy group, a trifluoroacetyl group, a triphenylmethyl group, a methoxydiphenylmethyl group, a 2,4,6-trimethoxybenzyl group, or a 2,2,4,6,7-pentamethyldihydrobenzofuran-5-sulfonyl group.

In one embodiment, an organic solvent may be used in the polymerization step.

In one embodiment, the organic solvent may include at least one selected from a group consisting of dioxane, dichloromethane, trichloromethane, tetrahydrofuran, methylbenzene, N, N-dimethylacetamide, N, N-dimethylformamide, dimethylsulfoxide, toluene, nitrobenzene, and N-methylpyrrolidone.

In one embodiment, the polymerization may be performed in an inert gas atmosphere.

In one embodiment, the method may further include, after the polymerization step, adding and reacting α-amino acid N-carboxyanhydride having the same structure as or a different structure from a structure of the α-amino acid N-carboxyanhydride used in the polymerization step.

In one embodiment, the method for producing the compound may produce the compound within 100 minutes.

In one embodiment, the compound produced using the method for producing the compound may have a polydispersity index (PDI) of 1.5 or lower.

A compound for achieving still another purpose of the present disclosure contains: a polymer ring structure formed using a compound represented by a following Chemical Formula 2 as a catalyst in a polymerization reaction of α-amino acid N-carboxyanhydride; and imidazole of the compound represented by the following Chemical Formula 2 bonded to the polymer ring structure while the imidazole shares a carbon atom constituting the polymer ring structure:

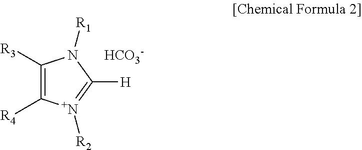

In the Chemical Formula 2, each of R₁ to R₄ independently represents a hydrogen atom, an alkyl group having 1 to 20 carbon atoms, a cycloalkyl group having 3 to 20 carbon atoms, ethylene glycol having 3 to 50 carbon atoms, an aryl group having 6 to 20 carbon atoms or a cycloalkenyl group having 5 to 20 carbon atoms.

A hydrogen atom of each of the alkyl group, the cycloalkyl group, the aryl group and the cycloalkenyl group may be independently substituted or unsubstituted with a substituent selected from a group consisting of an ether group, a carbonyl group, an alkenyl group, an allyl group, a halogen atom, a hydroxy group, a phenyl group, and a cyano group.

In one embodiment, the catalyst represented by the Chemical Formula 2 may include at least one of compounds represented by following Chemical Formulas 2-1, 2-2, 2-3 and 2-4.

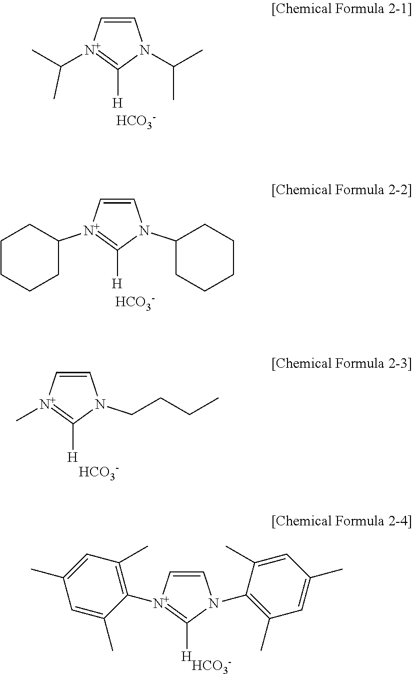

In one embodiment, the α-amino acid N-carboxyanhydride may be represented by a following Chemical Formula 3:

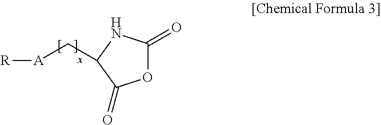

In the Chemical Formula 3, A represents a single bond, a hydrogen atom (—H—), a sulfur atom (—S—), an oxygen atom (—O—), a nitrogen atom (—N—),

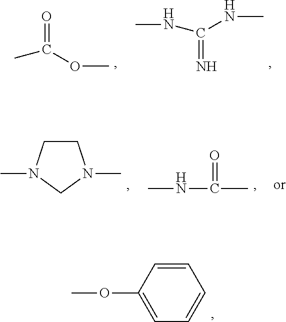

and R represents a hydrogen atom, a halogen atom, an alkyl group having 1 to 5 carbon atoms, an aryl group having 6 to 15 carbon atoms, a carbobenzoxy group, a trifluoroacetyl group, a carbonyl group, a triphenylmethyl group, a methoxydiphenylmethyl group, a 2,4,6-trimethoxybenzyl group, or a 2,2,4,6,7-pentamethyldihydrobenzofuran-5-sulfonyl group, where x represents an integer of 0 or greater.

In one embodiment, the α-amino acid N-carboxyanhydride may include at least one selected from a group consisting of protected or unprotected L-glycine N-carboxyanhydride, L-alanine N-carboxyanhydride, L-phenylalanine N-carboxyanhydride, L-valine N-carboxyanhydride, L-luecine N-carboxyanhydride, L-methlonine N-carboxyanhydride, L-isoleucine N-carboxyanhydride, L-proline N-carboxyanhydride, L-tryptophan N-carboxyanhydride, L-serine N-carboxyanhydride, L-cysteine N-carboxyanhydride, L-aspartic acid N-carboxyanhydride, L-glutamate N-carboxyanhydride, L-lysine N-carboxyanhydride, L-arginine N-carboxyanhydride, L-histidine N-carboxyanhydride, L-asparagine N-carboxyanhydride, L-glutamine N-carboxyanhydride, L-threonine N-carboxyanhydride, and L-tyrosine N-carboxyanhydride.

In one embodiment, the α-amino acid N-carboxyanhydride may include at least one of compounds represented by following Chemical Formulas A, B, C, D, E, F, G, H, I, J and K.

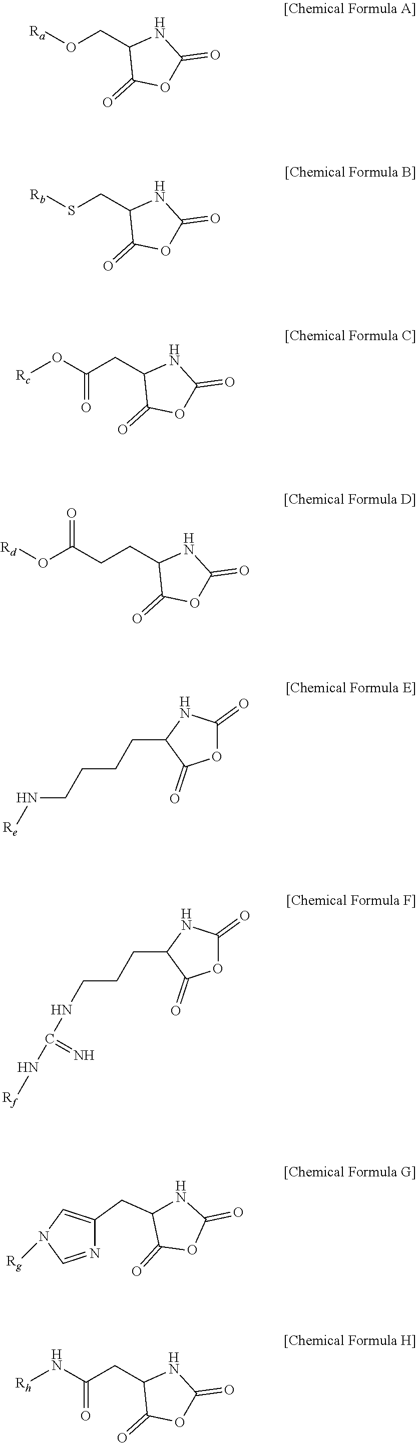

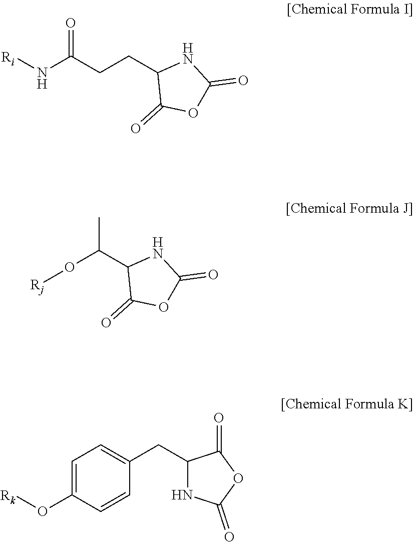

Each of Rₐ to Rₖ independently represents a hydrogen atom, a halogen atom, an alkyl group having 1 to 5 carbon atoms, an aryl group having 6 to 15 carbon atoms, a carbonyl group, a carbobenzoxy group, a trifluoroacetyl group, a triphenylmethyl group, a methoxydiphenylmethyl group, a 2,4,6-trimethoxybenzyl group, or a 2,2,4,6,7-pentamethyldihydrobenzofuran-5-sulfonyl group.

### Technical Effects

In accordance with the present disclosure, the cyclic compound containing the catalyst may be provided. A synthesis time duration may be reduced to about 100 minutes or smaller, while a conventional synthesis time duration is about three days or larger. Further, the method for producing the cyclic compound at a high yield while a molecular weight is controlled may be provided.

### BRIEF DESCRIPTION OF DRAWINGS

FIG. 1 to FIG. 9 show analysis results of compounds according to embodiments of the present disclosure.

### DETAILED DESCRIPTIONS

Hereinafter, the terminology used herein is for the purpose of describing particular embodiments only and is not intended to be limiting of the present disclosure. As used herein, the singular forms “a” and “an” are intended to include the plural forms as well, unless the context clearly indicates otherwise. It will be further understood that the terms “comprises”, “comprising”, “includes”, “including”, “haves” and “having” when used in this specification, specify the presence of the stated features, integers, operations, elements, and/or components, but do not preclude the presence or addition of one or more other features, integers, operations, elements, components, and/or portions thereof.

Unless otherwise defined, all terms including technical and scientific terms used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

A compound in accordance with the present disclosure is represented by a following Chemical Formula 1:

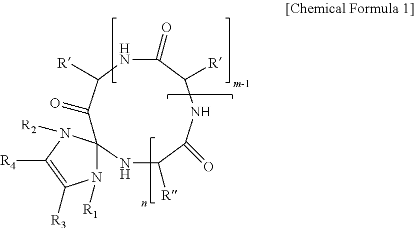

In the Chemical Formula 1, each of R₁ to R₄ independently represents a hydrogen atom, an alkyl group having 1 to 20 carbon atoms, a cycloalkyl group having 3 to 20 carbon atoms, an ethylene glycol group having 3 to 50 carbon atoms, an aryl group having 6 to 20 carbon atoms, or a cycloalkenyl group having 5 to 20 carbon atoms.

Each of R′ and R″ independently represents R-A-(CH₂)ₓ—*, where A represents a single bond, a sulfur atom (—S—), an oxygen atom (—O—), a nitrogen atom (—N—),

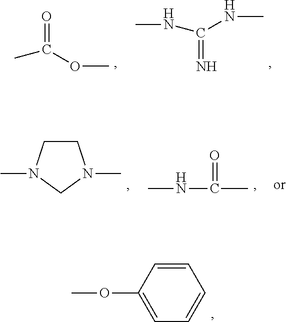

and R represents a hydrogen atom, a halogen atom, an alkyl group having 1 to 5 carbon atoms, an aryl group having 6 to 15 carbon atoms, a carbobenzoxy group, a trifluoroacetyl group, a carbonyl group, a triphenylmethyl group, a methoxydiphenylmethyl group, a 2,4,6-trimethoxybenzyl group, or a 2,2,4,6,7-pentamethyldihydrobenzofuran-5-sulfonyl group, where x represents an integer of 0 or greater.

A hydrogen atom of each of R₁ to R₄, R′ and R″ may be independently substituted or unsubstituted with a substituent selected from a group consisting of a halogen atom, a sulfur atom, an oxygen atom, a hydroxy group, an amine group, an ether group, a carbonyl group, an alkenyl group, an allyl group, a phenyl group, and a cyano group, where n is an integer greater than or equal to 0, and m is an integer of 1 or greater.

The alkyl group is defined as a functional group derived from a saturated hydrocarbon of a linear or branched structure. For example, specific examples of the alkyl group may include a methyl group, an ethyl group, a n-propyl group, an isopropyl group, an N-butyl group (normal-butyl group), a sec-butyl group, a tert-butyl group, an n-pentyl group, an N-octyl group (normal-octyl group), an n-decyl group, an n-hexadecyl group, a cyclopropyl group, a cyclopentyl group, a cyclohexyl group, a vinyl group, an allyl group, a 2-butenyl group, a 3-pentenyl group, a propargyl group, a 3-pentynyl group, and the like.

The cycloalkyl group represents a saturated hydrocarbon, that is, a substituent in a form of a ring consisting only of a carbon-carbon single bond. Specific examples thereof may include cyclopropane, cyclobutane, cyclopentane, cyclohexane, and the like.

The aryl group is defined as a monovalent substituent derived from an aromatic hydrocarbon. Specific examples of the aryl group may include a phenyl group, a naphtyl group, an anthracenyl group, a phenanathryl group, a naphthacenyl group, a pyrenyl group, a tolyl group, a biphenylyl group, a terphenyl group, a chrycenyl group, a spirobifluorene-yl group, a fluoranthene-yl group, a fluorenyl group,

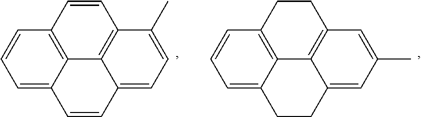

an indenyl group, an azulenyl group, a heptalenyl group, a phenalenyl group, a phenanthrenyl group, and the like.

In one embodiment, each of R₁ and R₂ may independently represent a cycloalkyl group, an alkyl group or an aryl group unsubstituted or substituted with the substituent.

In one embodiment, each of R₁ and R₂ may independently represent an alkyl group having 1 to 20 carbon atoms or a cycloalkyl group having 6 to 20 carbon atoms, and each of R₃ and R₄ may independently represent a hydrogen atom.

The compound according to the present disclosure may have a cyclic structure. The compound may be a large cyclic compound and may be, for example, a cyclic polypeptide, a block cyclic polypeptide or a macrocyclic polypeptide.

A method for producing a compound according to the present disclosure includes polymerizing α-amino acid N-carboxyanhydride using a catalyst represented by a following Chemical Formula 2:

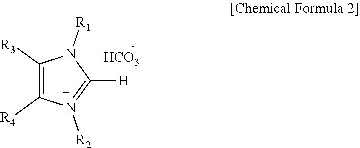

In the Chemical Formula 2, each of R₁ to R₄ independently represents a hydrogen atom, an alkyl group having 1 to 20 carbon atoms, a cycloalkyl group having 3 to 20 carbon atoms, ethylene glycol having 3 to 50 carbon atoms, an aryl group having 6 to 20 carbon atoms or a cycloalkenyl group having 5 to 20 carbon atoms. A hydrogen atom of each of the alkyl group, the cycloalkyl group, the aryl group and the cycloalkenyl group may be independently substituted or unsubstituted with a substituent selected from a group consisting of an ether group, a carbonyl group, an alkenyl group, an allyl group, a halogen atom, a hydroxy group, a phenyl group, and a cyano group.

The catalyst used in the method for producing the compound according to the present disclosure may include N-heterocyclic carbene. The N-heterocyclic carbene may be imidazole, and the imidazole may be present in a form of a salt that is stable in air. For example, the catalyst may be imidazolium carbonate.

The catalyst may be used as a catalyst for a ring-opening polymerization or a living polymerization.

Because the N-heterocyclic carbene exhibits high nucleophilicity, a time duration for which a side reaction such as a chain transfer reaction or a termination reaction that breaks a chain during the ring-opening polymerization is short. Thus, the side reaction such as the chain transfer reaction or the termination reaction may be suppressed.

The compound may contain α-amino acid N-carboxyanhydride. Since the α-amino acid N-carboxyanhydride has a living property, a molecular weight may be controlled based on a ratio between a monomer, an initiator and the catalyst. The α-amino acid N-carboxyanhydride may be about 20 or greater protected or unprotected amino acid carboxyanhydrides.

In one embodiment, each of R₁ and R₂ in the catalyst represented by the Chemical Formula 2 may independently represent a cycloalkyl group, an alkyl group or an aryl group unsubstituted or substituted with the substituent.

In one embodiment, the catalyst represented by the Chemical Formula 2 may include at least one of compounds represented by following Chemical Formulas 2-1, 2-2, 2-3 and 2-4.

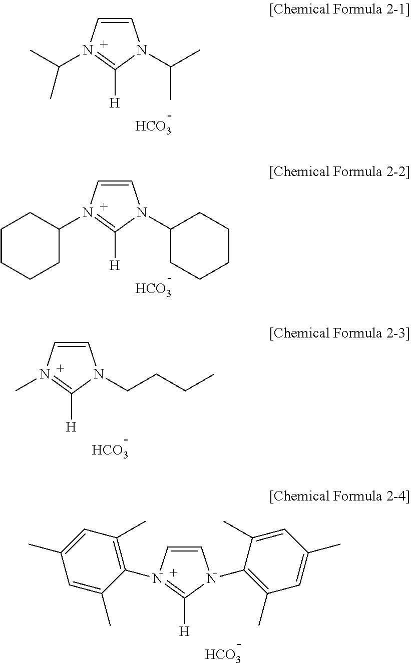

In one embodiment, the α-amino acid N-carboxyanhydride may be represented by a following Chemical Formula 3:

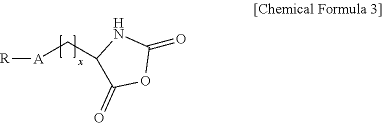

In the Chemical Formula 3, A represents a single bond, a hydrogen atom (—H—), a sulfur atom (—S—), an oxygen atom (—O—), a nitrogen atom (—N—),

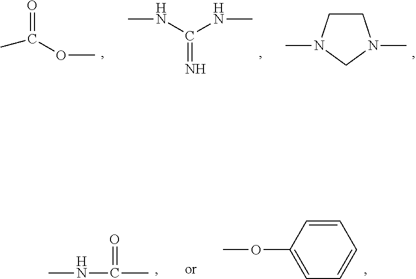

and R represents a hydrogen atom, a halogen atom, an alkyl group having 1 to 5 carbon atoms, an aryl group having 6 to 15 carbon atoms, a carbobenzoxy group, a trifluoroacetyl group, a carbonyl group, a triphenylmethyl group, a methoxydiphenylmethyl group, a 2,4,6-trimethoxybenzyl group, or a 2,2,4,6,7-pentamethyldihydrobenzofuran-5-sulfonyl group, where x represents an integer of 0 or greater.

In one embodiment, the α-amino acid N-carboxyanhydride may include at least one selected from a group consisting of protected or unprotected L-glycine N-carboxyanhydride, L-alanine N-carboxyanhydride, L-phenylalanine N-carboxyanhydride, L-valine N-carboxyanhydride, L-luecine N-carboxyanhydride, L-methlonine N-carboxyanhydride, L-isoleucine N-carboxyanhydride, L-proline N-carboxyanhydride, L-tryptophan N-carboxyanhydride, L-serine N-carboxyanhydride, L-cysteine N-carboxyanhydride, L-aspartic acid N-carboxyanhydride, L-glutamate N-carboxyanhydride, L-lysine N-carboxyanhydride, L-arginine N-carboxyanhydride, L-histidine N-carboxyanhydride, L-asparagine N-carboxyanhydride, L-glutamine N-carboxyanhydride, L-threonine N-carboxyanhydride, and L-tyrosine N-carboxyanhydride.

For example, the unprotected α-amino acid N-carboxyanhydride may include L-glycine N-carboxyanhydride, L-alanine N-carboxyanhydride, L-phenylalanine N-carboxyanhydride, L-valine N-carboxyanhydride, L-leucine N-carboxyanhydride, L-methionine N-carboxyanhydride, L-isoleucine N-carboxyanhydride, L-proline N-carboxyanhydride or L-tryptophan N-carboxyanhydride. The protected α-amino acid N-carboxyanhydride may include protected L-serine N-carboxyanhydride, protected L-cysteine N-carboxyanhydride, protected L-aspartic acid N-carboxyanhydride, protected L-glutamate N-carboxyanhydride, protected L-lysine N-carboxyanhydride, protected L-arginine N-carboxyanhydride, protected L-histidine N-carboxyanhydride, protected L-asparagine N-carboxyanhydride, protected L-glutamine N-carboxyanhydride, protected L-threonine N-carboxyanhydride or protected L-tyrosine N-carboxyanhydride.

In one embodiment, the α-amino acid N-carboxyanhydride may include at least one of compounds represented by following Chemical Formulas A, B, C, D, E, F, G, H, I, J and K.

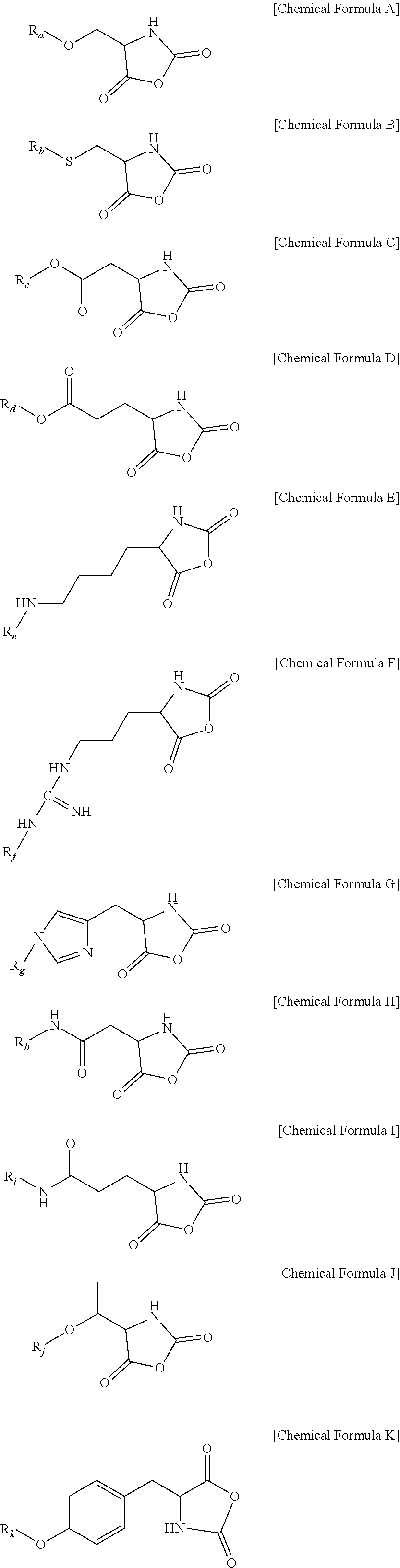

Each of Rₐ to Rₖ independently represents a hydrogen atom, a halogen atom, an alkyl group having 1 to 5 carbon atoms, an aryl group having 6 to 15 carbon atoms, a carbonyl group, a carbobenzoxy group, a trifluoroacetyl group, a triphenylmethyl group, a methoxydiphenylmethyl group, a 2,4,6-trimethoxybenzyl group, or a 2,2,4,6,7-pentamethyldihydrobenzofuran-5-sulfonyl group.

The protected L-serine N-carboxyanhydride may be represented by the Chemical Formula A. Rₐ in the Chemical Formula A may be a methyl group, an ethyl group, a benzyl group, or a benzyl group substituted with one or more of a tert-butyl group, an allyl group, a halogen atom, and the like.

The protected L-cysteine N-carboxyanhydride may be represented by the Chemical Formula B. R_{b} in the Chemical Formula B may be a benzyl group, a tert-butyl group, or a 4-methyl benzyl group.

The protected L-aspartic acid N-carboxyanhydride may be represented by the Chemical Formula C. R_{c} in the Chemical Formula C may be a methyl group, an ethyl group, a benzyl group or a benzyl group substituted with one or more of a tert-butyl group, an allyl group, a halogen atom, and the like.

The protected L-glutamate N-carboxyanhydride may be represented by the Chemical Formula D. R_{d} in the Chemical Formula D may be a methyl group, an ethyl group, a benzyl group or a benzyl group substituted with one or more of a tert-butyl group, an allyl group, a halogen atom, and the like.

The protected L-lysine N-carboxyanhydride may be represented by the Chemical Formula E. Rₑ in the Chemical Formula E may be a carbobenzoxy group, a trifluoroacetyl group, a t-butyloxy carbonyl group, or an alioxycarbonyl group and the like.

The protected L-arginine N-carboxyanhydride may be represented by the Chemical Formula F. In the Chemical Formula F, R_{f} may be 2,2,4,6,7-pentamethyldihydrobenzofuran-5-sulfonyl group or an alioxycarbonyl group.

The protected L-histidine N-carboxyanhydride may be represented by the Chemical Formula G. R_{g} in the Chemical Formula G may be a benzyl group or a t-butyloxy carbonyl group and the like.

The protected L-asparagine N-carboxyanhydride may be represented by the Chemical Formula H. In the Chemical Formula H, Rₕ may be a triphenylmethyl group, a 2,4,6-trimethoxybenzyl group, a methoxydiphenylmethyl group or an alioxycarbonyl group.

The protected L-glutamine N-carboxyanhydride may be represented by the Chemical Formula I. Rᵢ in the Chemical Formula I may be a triphenylmethyl group, a 2,4,6-trimethoxybenzyl group, a methoxydiphenylmethyl group or an alioxycarbonyl group.

The protected L-threonine N-carboxyanhydride may be represented by the Chemical Formula J. Rⱼ in the Chemical Formula J may be a methyl group, an ethyl group, a benzyl group, or a benzyl group substituted with one or more of a tert-butyl group, an allyl group, a halogen atom, and the like.

The protected L-tyrosine N-carboxyanhydride may be represented by the Chemical Formula K. In the Chemical Formula K, Rₖ may be a methyl group, an ethyl group, a benzyl group, or a benzyl group substituted with one or more of a tert-butyl group, an allyl group, a halogen atom, and the like.

An organic solvent may be used in the polymerization step of the method for producing the compound according to the present disclosure. In one embodiment, the organic solvent may include at least one selected from a group consisting of dioxane, dichloromethane, trichloromethane, tetrahydrofuran, methylbenzene, N, N-dimethylacetamide, N, N-dimethylformamide, dimethylsulfoxide, toluene, nitrobenzene, and N-methylpyrrolidone. For example, as the organic solvent, the above components may be used alone or in combination of two or more thereof. For example, as the organic solvent, dimethylformamide may be used alone.

The polymerization may be performed in an inert gas atmosphere. For example, the inert gas may be argon or nitrogen gas, or a combination of the two.

In one embodiment, the method may further include, after the polymerization step, adding and reacting α-amino acid N-carboxyanhydride having the same structure as or a different structure from a structure of the α-amino acid N-carboxyanhydride used in the polymerization step. The α-amino acid N-carboxyanhydride further added after the polymerization may be α-amino acid N-carboxyanhydride which is not the same as the α-amino acid N-carboxyanhydride used in the polymerization.

The method for producing the compound may produce the compound within 100 minutes.

In this connection, this means that an entire process of producing the compound is performed within 100 minutes. The method for producing the compound may be performed, for example, for about 5 to 100 minutes. The compound produced using the method for producing the compound according to the present disclosure may contain a peptide bond, and the compound may contain a polypeptide. Therefore, a cyclic polypeptide may be produced using the method for producing the compound according to the present disclosure. The polymerization time duration of the cyclic polypeptide based compound which took about 3 days or larger conventionally may be shortened to about 5 to 100 minutes or smaller in accordance with the present disclosure. As the polymerization time duration is shortened, a molecular weight distribution of the compound as produced is narrowed, thereby achieving excellent physical properties.

The compounds produced using the method for producing the compound according to the present disclosure may have a polydispersity index (PDI) of about 1.5 or lower. The polydispersity means that molecular properties of a polymer compound are nonuniform. A typical example of the molecular properties is a molecular weight distribution. The polydispersity is opposite to monodispersity. For example, the compound has a polydispersity index lower than or equal to about 1.3. The closer the PDI value is to 1, the more monodisperse the compound is. As the PDI value is increasingly larger than 1, the compound is more polydisperse. Therefore, the closer the PDI value of the polymer to 1, the narrower the molecular weight distribution, and the better the physical properties.

A molar content of the amino acid anhydride with respect to 1 mole of the catalyst in the polymerization step of the method according to the present disclosure may be in a range of about 5 to 2000 moles or of about 5 to 800 moles, preferably, of about 10 to 500 moles. The compounds produced via the polymerization may be represented by a following Chemical Formula 4, where, n′ is an integer of 1 or greater:

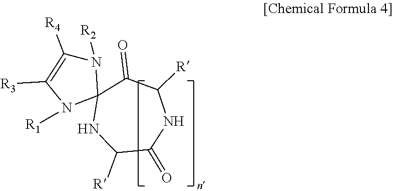

The polymerization step may be represented by a following Reaction Formula 1, where n′ means an integer of 1 or greater:

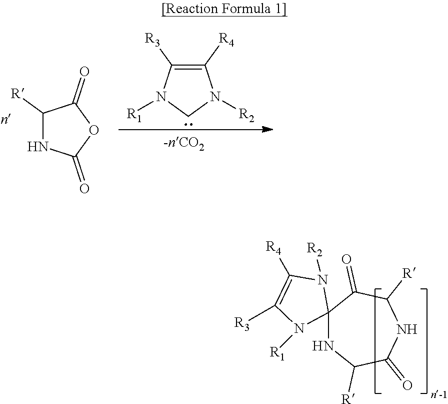

In order to produce the compound represented by the Chemical Formula 4, α-amino acid N-carboxyanhydride is dissolved in the organic solvent under a nitrogen atmosphere to form a mixed solution. Then, the catalyst is added to the mixed solution which is then polymerized to obtain the compound represented by the Chemical Formula 4.

Addition of an initiator in the polymerization step may produce the compound having a linear structure rather than a cyclic structure. A reaction using the initiator may be represented by a following Reaction Formula 2, where n′ is an integer of 1 or greater:

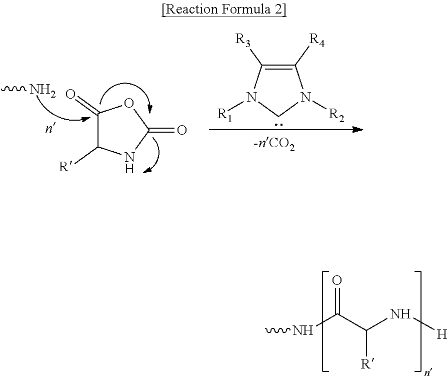

In order to produce the compound having a linear structure, α-amino acid N-carboxyanhydride is dissolved in the organic solvent under a nitrogen atmosphere to form a mixed solution. Then, the initiator and the catalyst are added to the mixed solution which is then polymerized to obtain the compound having a linear structure.

A molar ratio between the catalyst, the initiator and the α-amino acid N-carboxyanhydride may be configured such that a content of the initiator may be 0.2 to 10 moles based on 1 mole of the catalyst, and a content of the α-amino acid N-carboxyanhydride may be 2 to 10000 moles based on 1 mole of the catalyst. For example, the content of the initiator may be 0.5 to 2 moles and the content of the α-amino acid N-carboxyanhydride may be 10 to 200 moles based on 1 mole of the catalyst.

A primary amine may be used as the initiator. For example, the initiator may employ at least one selected from a group consisting of n-butylamine, n-amylamine, n-hexylamine, diethylamine, triethylamine, imidazole, hexamethyl-disilazane, phenylamine, benzylamine, benzylethylamine, phosphatidylethanolamine, silazane derivatives such as (trimethylsilyl)methanamine or (trimethoxysilyl)methanamine, amine trifluoroborane, amine hydrochlorides, phosphatidylethanolamine, mono methoxy polyethylene glycol amine, and macroinitiator.

After the main or previous polymerizing step, the α-amino acid N-carboxyanhydride is further added. Then, a subsequent polymerizing step may occur and may be represented by a following Reaction Formula 3, where n′ is an integer of 1 or greater, and m is an integer of 1 or greater:

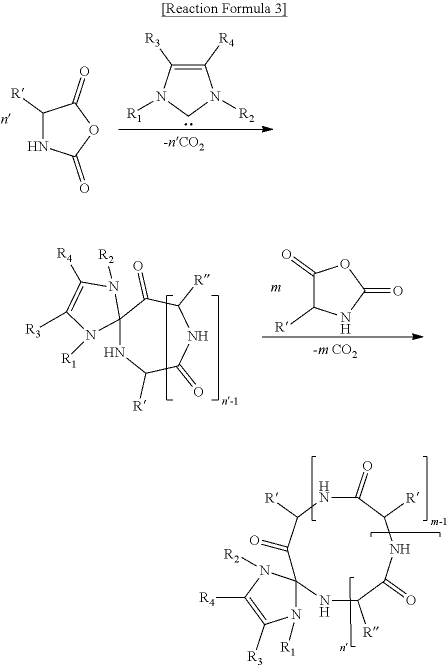

After the main or previous polymerization step for producing the compound according to the present disclosure, the addition of the α-amino acid N-carboxyanhydride and then the subsequent polymerization may be performed. In this connection, the method may further add the α-amino acid N-carboxyanhydride to the compound produced in the previous polymerization step and then perform the subsequent polymerization. The α-amino acid N-carboxyanhydride in the subsequent polymerization may be different from the α-amino acid N-carboxyanhydride in the previous polymerization. The subsequent polymerizing step may be performed at a temperature of about 10 to 50° C., for example, at room temperature. The subsequent polymerizing step may be performed for about 5 to 100 minutes.

When the initiator is used in the main or previous polymerization step, the compound having a linear structure may be produced. Subsequently, when the subsequent polymerization is carried out, the compound having a longer linear structure may be produced. This process may be represented as a following Reaction Formula 4, where n′ is an integer of 1 or greater, and m is an integer of 1 or greater:

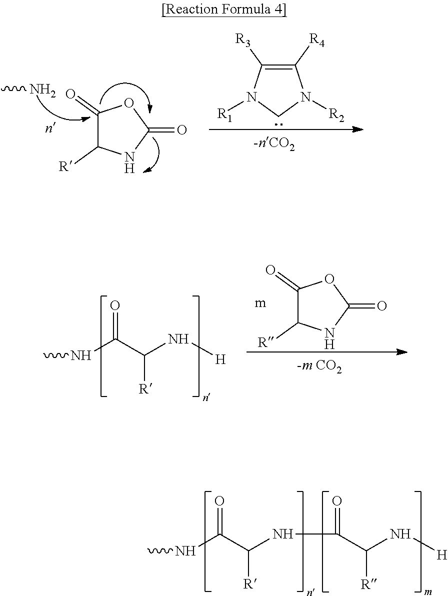

In the method for producing the compound according to the present disclosure, the polymerization may be a ring-opening polymerization or a living polymerization.

A compound in accordance with the present disclosure may contain: a polymer ring structure formed using a compound represented by a following Chemical Formula 2 as a catalyst in a polymerization reaction of α-amino acid N-carboxyanhydride; and imidazole of the compound represented by the following Chemical Formula 2 bonded to the polymer ring structure while the imidazole shares a carbon atom constituting the polymer ring structure:

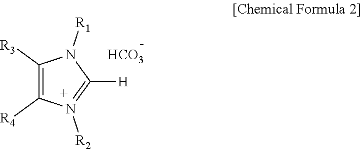

In the Chemical Formula 2, each of R₁ to R₄ independently represents a hydrogen atom, an alkyl group having 1 to 20 carbon atoms, a cycloalkyl group having 3 to 20 carbon atoms, ethylene glycol having 3 to 50 carbon atoms, an aryl group having 6 to 20 carbon atoms or a cycloalkenyl group having 5 to 20 carbon atoms. A hydrogen atom of each of the alkyl group, the cycloalkyl group, the aryl group and the cycloalkenyl group may be independently substituted or unsubstituted with a substituent selected from a group consisting of an ether group, a carbonyl group, an alkenyl group, an allyl group, a halogen atom, a hydroxy group, a phenyl group, and a cyano group.

The catalyst may include imidazole and may have a moiety having a cation and a moiety having an anion. The moiety having the cation of the catalyst may be bonded to the polymer ring structure, or the imidazole of the catalyst may be bonded to the polymer ring structure. The imidazole of the catalyst may be embodied as the cationic moiety and the carbonate may be embodied as the anionic moiety. The catalyst may have a form of an imidazole ring containing two nitrogen atoms. One carbon atom may be located between the two nitrogen atoms that constitute the imidazole ring.

The compound according to the present disclosure is produced via the polymerization reaction of the α-amino acid N-carboxyanhydride. In the polymerization reaction, the compound represented by the Chemical Formula 2 according to the present disclosure may be used as the catalyst. The catalyst may be used for the polymerization of the α-amino acid N-carboxyanhydride to form the polymer ring structure. The imidazole contained in the catalyst may be bound to the polymer ring structure. The polymer ring structure and the imidazole may be bonded to each other while both share one carbon atom constituting the polymer ring structure and one carbon atom constituting the imidazole with each other.

The compound has a structure in which the compound represented by the Chemical Formula 2 is bonded to the cyclic peptide. The cyclic peptide may be produced via the polymerization of the α-amino acid N-carboxyanhydride using the catalyst as the compound represented by the Chemical Formula 2. In other words, the cyclic peptide may be produced via the polymerization of the α-amino acid N-carboxyanhydride. The cationic moiety of the compound represented by the Chemical Formula 2 used as the catalyst in the polymerization reaction may be bound to the cyclic peptide.

The catalyst represented by the Chemical Formula 2 may be used in the living polymerization reaction or the ring-opening polymerization reaction of the α-amino acid N-carboxyanhydride to form the polymer ring structure.

The catalyst and the polymer ring structure may share at least one carbon atom. The catalyst and the polymer ring structure may be bonded to each other while both share the carbon atom. The shared carbon atom may be one carbon atom located between the two nitrogen atoms constituting the imidazole ring, or may be one carbon atom located between the nitrogen atom constituting the polymer ring structure and the carbonyl group. The imidazole may be bonded to the polymer ring structure while one carbon atom located between the two nitrogen atoms is bonded in a covalent bond manner to one carbon atom constituting the polymer ring structure. In other words, the shared carbon may be located between peptide bonds constituting the polymer ring structure. Alternatively, the carbon atom located between the two nitrogen atoms constituting the imidazole ring may be inserted between the peptide bonds constituting the polymer ring structure. The shared carbon atom of the compound according to the present disclosure may be due to the bonding between three nitrogen atoms and one carbon atom.

The compound according to the present disclosure may have a polymer ring structure containing the imidazole as a carbene compound. In this connection, the carbene compound including the imidazole may be used as the catalyst in the process of producing the compound according to the present disclosure. The compound according to the present disclosure may be a cyclic compound containing the catalyst bonded to a compound produced using the catalyst used in the polymerization reaction for producing the compound. For example, the cyclic compound may be a cyclic polypeptide.

The compound may be represented by the Chemical Formula 1 in which the imidazole ring and the polymer ring structure are bonded to each other:

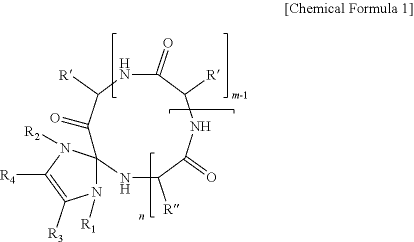

In the Chemical Formula 1, each of R₁ to R₄ independently represents a hydrogen atom, an alkyl group having 1 to 20 carbon atoms, a cycloalkyl group having 3 to 20 carbon atoms, an ethylene glycol group having 3 to 50 carbon atoms, an aryl group having 6 to 20 carbon atoms, or a cycloalkenyl group having 5 to 20 carbon atoms. Each of R′ and R″ independently represents R-A-(CH₂)ₓ—*, where A represents a single bond, a sulfur atom (—S—), an oxygen atom (—O—), a nitrogen atom (—N—),

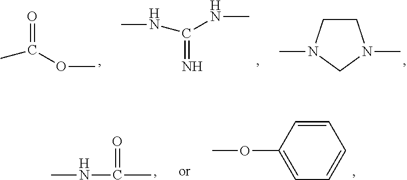

and R represents a hydrogen atom, a halogen atom, an alkyl group having 1 to 5 carbon atoms, an aryl group having 6 to 15 carbon atoms, a carbobenzoxy group, a trifluoroacetyl group, a carbonyl group, a triphenylmethyl group, a methoxydiphenylmethyl group, a 2,4,6-trimethoxybenzyl group, or a 2,2,4,6,7-pentamethyldihydrobenzofuran-5-sulfonyl group, where x represents an integer of 0 or greater. A hydrogen atom of each of R₁ to R₄, R′ and R″ may be independently substituted or unsubstituted with a substituent selected from a group consisting of a halogen atom, a sulfur atom, an oxygen atom, a hydroxy group, an amine group, an ether group, a carbonyl group, an alkenyl group, an allyl group, a phenyl group, and a cyano group, where n is an integer greater than or equal to 0, and m is an integer of 1 or greater.

In one embodiment, the catalyst represented by the Chemical Formula 2 may include at least one of compounds represented by following Chemical Formulas 2-1, 2-2, 2-3 and 2-4.

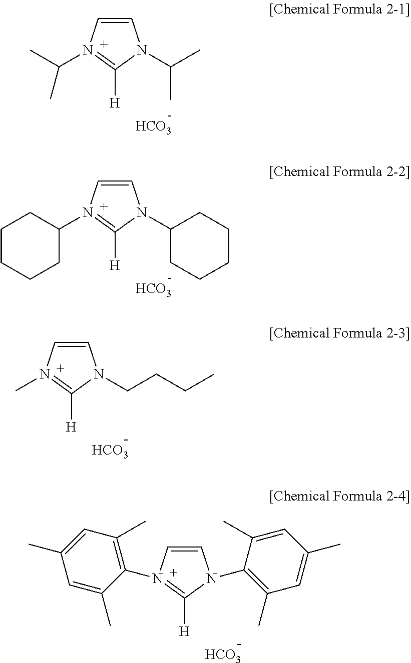

In one embodiment, the α-amino acid N-carboxyanhydride may be represented by a following Chemical Formula 3:

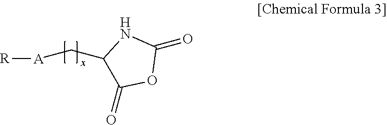

In the Chemical Formula 3, A represents a single bond, a hydrogen atom (—H—), a sulfur atom (—S—), an oxygen atom (—O—), a nitrogen atom (—N—)

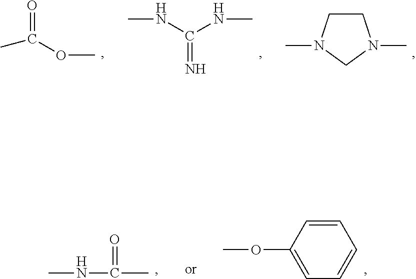

and R represents a hydrogen atom, a halogen atom, an alkyl group having 1 to 5 carbon atoms, an aryl group having 6 to 15 carbon atoms, a carbobenzoxy group, a trifluoroacetyl group, a carbonyl group, a triphenylmethyl group, a methoxydiphenylmethyl group, a 2,4,6-trimethoxybenzyl group, or a 2,2,4,6,7-pentamethyldihydrobenzofuran-5-sulfonyl group, where x represents an integer of 0 or greater.

In one embodiment, the α-amino acid N-carboxyanhydride may include at least one selected from a group consisting of protected or unprotected L-glycine N-carboxyanhydride, L-alanine N-carboxyanhydride, L-phenylalanine N-carboxyanhydride, L-valine N-carboxyanhydride, L-luecine N-carboxyanhydride, L-methlonine N-carboxyanhydride, L-isoleucine N-carboxyanhydride, L-proline N-carboxyanhydride, L-tryptophan N-carboxyanhydride, L-serine N-carboxyanhydride, L-cysteine N-carboxyanhydride, L-aspartic acid N-carboxyanhydride, L-glutamate N-carboxyanhydride, L-lysine N-carboxyanhydride, L-arginine N-carboxyanhydride, L-histidine N-carboxyanhydride, L-asparagine N-carboxyanhydride, L-glutamine N-carboxyanhydride, L-threonine N-carboxyanhydride, and L-tyrosine N-carboxyanhydride.

In one embodiment, the α-amino acid N-carboxyanhydride may include at least one of compounds represented by the above Chemical Formulas A, B, C, D, E, F, G, H, I, J and K. Each of Rₐ to Rₖ independently represents a hydrogen atom, a halogen atom, an alkyl group having 1 to 5 carbon atoms, an aryl group having 6 to 15 carbon atoms, a carbonyl group, a carbobenzoxy group, a trifluoroacetyl group, a triphenylmethyl group, a methoxydiphenylmethyl group, a 2,4,6-trimethoxybenzyl group, or a 2,2,4,6,7-pentamethyldihydrobenzofuran-5-sulfonyl group.

When using an imidazolium carbonate compound as the catalyst in accordance with the present disclosure, a reaction may be more stable and polymerization efficiency and polymerization rate may be further improved compared to a case when using other carbene compounds as the catalyst. At the same time, the method for producing the compound according to the present disclosure may produce not only a low molecular weight cyclic compound but also a high molecular weight cyclic compound in a faster manner than the conventional techniques may produce. The molecular weight of the compound as produced may be controlled, and the molecular weight distribution may be controlled to be narrower in accordance with the present disclosure.

The compound produced via the method for producing the compound according to the present disclosure may include linear and cyclic polypeptides. Conventional techniques for producing a polypeptide using a metal catalyst exist. However, when using the metal catalyst, there was a problem that the compound may not be used as a biomaterial, and that it takes too long time to produce the polypeptide. However, the catalyst represented by the Chemical Formula 2 according to the present disclosure does not contain the metal. Thus, when using the present catalyst, the produced polypeptide may be applied to the biotechnology field without the above problem.

Hereinafter, Examples of the present disclosure will be described in detail. However, the following Examples are only some embodiments of the present disclosure, and the present disclosure should not be construed as being limited to the following Examples.

### Example: [Chemical Formula 2] Producing (Catalyst Producing)

In Example, the catalyst in accordance with the present disclosure may be synthesized.

### 1,3-diisopropy imidazolium hydrogen carbonate (2-1) Producing

First, under nitrogen atmosphere, about 500 mg (about 2.14 mmol) of 1,3-diisopropylimidazolium chloride was input to a schlenk tube in which oxygen was removed and which was dried, and 1.2 eq of dry potassium bicarbonate (KHCO₃) was added thereto. About 5 mL methanol was added thereto. A mixture was stirred to form a suspension. Subsequently, the mixture was reacted for about 48 hours at room temperature in a nitrogen atmosphere, and then was filtered through a glass filter to obtain a clear solution. The solution was dried in vacuum and washed with acetone and dried for a short time to obtain 1,3-diisopropyl imidazolium carbonate catalyst represented by [Chemical Formula 2-1] (yield: about 77%).

We further produced three catalysts with different structures using the method of the Example. Thus, four catalysts 2-1, 2-2, 2-3 and 2-4 were produced and are shown in Table 1:

TABLE 1

| | |
|---|---|
| 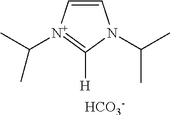 | [Chemical Formula 2-1] |
| 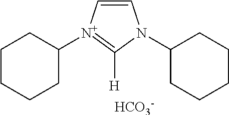 | [Chemical Formula 2-2] |
| 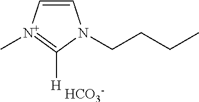 | [Chemical Formula 2-3] |
| 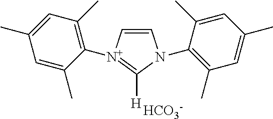 | [Chemical Formula 2-4] |

### Example: Compound Producing

### Example 1. γ-benzyl L-glutamate N-carboxyanhydride (Bn-Glu-NCA) Producing

First, about 2.37 g (about 10 mmol) of H-Glu(OBzl)-OH and about 40 mL tetrahydrofuran were added to a dried 250 mL schlenk tube in which nitrogen was removed. Then, about 1.49 g of triphosgene was dissolved in about 10 mL thereof while being slowly added thereto. A mixture was stirred at about 40° C. for suspension. A reaction was terminated when the suspended solution became clear. A clear solution was obtained within about 2 hours. After the polymerization reaction, the solvent was cooled and then was bubbled using nitrogen, and an unreacted material was removed using phosgene and hydrochloric acid (HCl). The polymerized solution was then concentrated under high vacuum. The concentrated reaction solution was precipitated in excessive hexane and was filtered through a glass filter to obtain Bn-Glu-NCA. The product was then rinsed and dried in vacuum at about 50° C.

Using the method of the Example, ε-carbobenzoxy-L-lysine N-carboxyanhydride, L-alanine N-carboxyanhydride, L-leucine N-carboxyanhydride, S-benzyl-L-cysteine N-carboxyanhydride, L-phenylalanine N-carboxyanhydride, and the like were synthesized.

### Example 2. Cyclic poly(γ-benzyl L-glutamate) Producing

About 131.63 mg (about 5.0×10⁴ mol) of γ-benzyl L-glutamate produced in the Example was added to an oxygen-depleted and dried schlenk tube under nitrogen atmosphere. Then, about 1.5 mL dimethylformamide (DMF) was added thereto for dissolution. N-heterocyclic carbene (NHC) 1/DMF mother liquor (about 500 μL, about 1×10⁻⁵ mol, about 0.02 M) was added thereto using a syringe to produce a mixture. The mixture was reacted for about 30 minutes at room temperature under a nitrogen atmosphere. The solution was then precipitated in methanol, and was filtered and was dried in vacuum to form cyclic poly(γ-benzyl L-glutamate).

To compare a conversion, a molecular weight and a polydispersity index while changing the α-amino acid N-carboxyanhydride (NCA) type, a molar ratio between the monomer and the catalyst, and a time condition, Examples 3 to 10 were performed while changing the conditions of the Example 2. Results of Examples 3 to 10 are shown in Table 2 below. It may be identified that the closer the PDI value of the polymer is to 1, the better the physical properties. The conversion shown in Table 2 is expressed in term of a percentage.

When comparing Examples 3 to 10 with each other, in Example 9 in which a content of benzyl glutamate was about 100 moles based on 1 mole of the catalyst, the polymerization was carried out for a reaction time of about 10 minutes. it was confirmed that as a result of the polymerization, the conversion was about 97%, and a high molecular weight distribution approximate to monodispersion was achieved.

TABLE 2

| | | Monomer:catalyst | Time | Conversion | Mn | |
|---|---|---|---|---|---|---|
| Examples | NCA type | (molar ratio) | (min) | (%) | (kg/mol) | PDI |
| Example 3 | Bn-Cys | 20:1 | 5 | 28 | 1.1 | — |
| Example 4 | Phe | 20:1 | 6 | 55 | 1.7 | — |
| Example 5 | Ala | 10:1 | 10 | 75 | 0.65 | — |
| Example 6 | Ala | 50:1 | 9 | 78 | 2.9 | — |
| Example 7 | Bn-Glu | 10:1 | 10 | 100 | 2.34 | 1.15 |
| Example 8 | Bn-Glu | 80:1 | 30 | 100 | 19.4 | 1.18 |
| Example 9 | Bn-Glu | 100:1 | 10 | 97 | 21.4 | 1.18 |
| Example 10 | Z-Lys | 100:1 | 20 | 81 | 21.2 | 1.30 |

The Bn-Cys represents benzyl cysteine carboxyanhydride, the Phe represents phenylalanine carboxyanhydride, the Ala represents alanine carboxyanhydride, Bn-Glu represents benzyl glutamate carboxyanhydride, and Z-Lys represents carbobenzoxy lysine carboxyanhydride. The PDI of the Examples was measured using SEC, but PDI of the polypeptide that was not dissolved in DMF was not measured. As shown in the Examples of the table herein, small cyclic or macrocyclic peptides may be obtained by adjusting the molar ratios between the monomer and the catalyst of the cyclic peptide. For example, as shown in the Table 2, regarding the molar ratio of the monomer and the catalyst, when the monomer content is 10 to 20 moles based on 1 mole of the catalyst (monomer:catalyst=10:1 or 20:1), a small cyclic peptide may be obtained. As shown in the Table 2, regarding the molar ratio of the monomers and the catalyst, when the monomer content is 50 to 80 moles based on 1 mole of the catalyst (monomer:catalyst=50:1 or 80:1), a macro cyclic peptide may be obtained. In addition, when controlling the molar ratio between the monomer, the initiator, and the catalyst (monomer:initiator:catalyst), macrocyclic peptides may be obtained from linear peptides.

### Example 11. Linear poly(γ-benzyl L-glutamate) Producing

About 263.25 mg (about 1 mmol) of γ-benzyl L-glutamate was added to an oxygen-removed and dried schlenk tube under a nitrogen atmosphere. Then, about 4.5 mL dimethylformamide (DMF) was added thereto for dissolution. Then about 2.7 μL (about 2×10⁻⁵ mol) n-hexylamine was added thereto. The heterocyclic carbene 1/DMF mother liquor (about 500 μL, 1×10⁻⁵, about 0.02 M) was added thereto using a syringe to produce a mixture. The mixture was reacted for about 30 minutes at room temperature under a nitrogen atmosphere. After the polymerization reaction, the solution was precipitated in methanol and filtered and dried in vacuum to produce linear poly(γ-benzyl L-glutamate).

To compare a conversion, a molecular weight and a polydispersity index while changing the α-amino acid N-carboxyanhydride (NCA) type, a molar ratio between the monomer and the catalyst, and a time condition, Examples 12 to 26 were performed while changing the conditions of the Example 11. Results of Examples 12 to 26 are shown in Table 3 below. Based on 1 mole of the initiator, 10, 50, 80 and 100 moles of the monomers and 0.2 mole of the catalyst were used respectively. The reaction was performed for about 5 to 30 minutes. In the Example 21 to 25 using benzyl glutamate carboxyanhydride as the monomer, the reaction was performed while changing a type of the catalyst (using catalysts 2-2, 2-3 and 2-4) and changing the molar content of the monomer. In this connection, in Examples 22 to 25 except for Example 21 where the reaction time was smaller than 10 minutes, the conversion was about 95% or greater. The polydispersity index thereof was close to 1, thus indicating that the molecular weight distribution is narrow.

TABLE 3

| | | | | | Time | Conversion | Mn | |
|---|---|---|---|---|---|---|---|---|
| Examples | NCA | Initiator | Catalyst | Molar ratio | (min) | (%) | (kg/mol) | PDI |
| Example 12 | Ala | BnA | 2-1 | 50:1:0.2 | 5 | 53 | 1.9 | — |
| Example 13 | Leu | BnA | 2-1 | 50:1:0.2 | 5 | 42 | 2.4 | — |
| Example 14 | Phe | Hxa | 2-1 | 10:1:0.2 | 10 | 82 | 1.2 | — |
| Example 15 | Phe | HxA | 2-1 | 50:1:0.2 | 8 | 64 | 4.7 | — |
| Example 16 | Bn-Cys | OtA | 2-1 | 10:1:0.2 | 8 | 78 | 1.5 | — |
| Example 17 | Bn-Cys | OtA | 2-1 | 50:1:0.2 | 8 | 67 | 6.5 | — |
| Example 18 | Z-Lys | HxA | 2-1 | 10:1:0.2 | 15 | 98 | 2.6 | 1.22 |
| Example 19 | Z-Lys | HxA | 2-1 | 50:1:0.2 | 20 | 99 | 13.3 | 1.30 |
| Example 20 | Z-Lys | PE | 2-1 | 50:1:0.2 | 30 | 95 | 13.1 | — |
| Example 21 | Bn-Glu | HxA | 2-1 | 10:1:0.2 | 6 | 78 | 8.5 | 1.17 |
| Example 22 | Bn-Glu | HxA | 2-1 | 80:1:0.2 | 30 | 97 | 17.2 | 1.07 |
| Example 23 | Bn-Glu | HxA | 2-1 | 100:1:0.2 | 10 | 98 | 21.5 | 1.20 |
| Example 24 | Bn-Glu | HxA | 2-2 | 100:1:0.2 | 10 | 98 | 21.5 | 1.19 |
| Example 25 | Bn-Glu | HxA | 2-3 | 100:1:0.2 | 10 | 96 | 21.0 | 1.20 |
| Example 26 | Bn-Glu | HxA | 2-4 | 100:1:0.2 | 10 | 97 | 21.3 | 1.25 |

The Ala stands for alanine carboxyanhydride, the Leu stands for leucine carboxyanhydride, and the Phe stands for phenylalanine carboxyanhydride. The Bn-Cys represents benzyl cysteine carboxyanhydride, Z-Lys represents carbobenzoxylysine carboxyanhydride and Bn-Glu represents benzyl glutamate carboxyanhydride. Further, the BnA stands for benzylamine, the HxA stands for hexylamine, and the PE stands for phosphatidylethanolamine. The PDI of the Examples was measured using SEC, but the PDI of the polypeptide that was not dissolved in DMF was not measured.

### Example: Block Compound Producing

### Example 27. Production of Block cyclic poly(γ-benzyl L-glutamate)-b-poly(ε-carbobenzoxy-L-lysine)

Cyclic poly(γ-benzyl L-glutamate) ([M₁]₀=0.2 M, [M₁]₀/[NHC]₀=30/1) was synthesized as in the above Example 9. Then, lysine N-carboxyanhydride ([M₂]₀/[NHC]₀=70/1) dissolved in about 2 mL of dimethylformamide was added to cyclic poly(γ-benzyl L-glutamate) using a syringe to produce a mixture. The mixture was reacted for about 30 minutes at room temperature under a nitrogen atmosphere. After the polymerization reaction, the solution was precipitated in methanol and was filtered and dried in vacuum to produce block cyclic poly(γ-benzyl L-glutamate)-b-poly(ε-carbobenzoxy-L-lysine).

### Example 28. Production of block linear poly(γ-benzyl L-glutamate)-b-poly(ε-carbobenzoxy-L-lysine)

Linear poly(γ-benzyl L-glutamate) ([M₁]₀=0.2 M, [M₁]₀/[NHC]₀=20:1, [NHC]₀=2.0 mM) was synthesized as in the above Example 11. Then, Z-lysine N-carboxyanhydride ([M₂]₀/[NHC]₀=60/1) dissolved in about 2 mL of dimethylformamide was added to the linear poly(γ-benzyl L-glutamate) using a syringe to produce a mixture. The mixture was reacted for about 30 minutes at room temperature under a nitrogen atmosphere. After the polymerization reaction, the solution was precipitated in methanol, filtered and dried in vacuum to obtain linear poly(γ-benzyl L-glutamate)-b-poly(ε-carbobenzoxy-L-lysine).

Characteristic Evaluation

MALDI-TOF MS Measurement

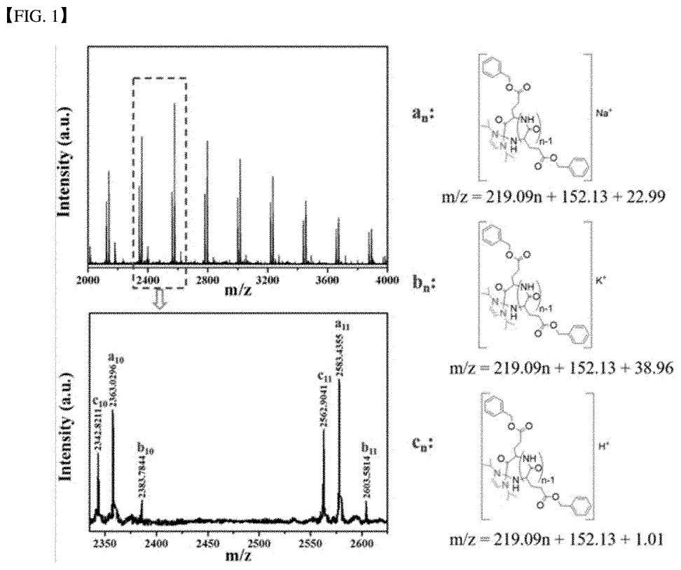

FIG. 1 shows a result of compound analysis. Specifically, FIG. 1 shows mass spectrometric analysis of small-molecular-weight cyclic poly(γ-benzyl L-glutamate) as produced according to the present disclosure. The mass spectrometry data was measured using DIT matrix (matrix-assisted laser desorption/ionization time-of-flight mass spectrometry (MALDI-TOF MS)). The mass spectrometry data in FIG. 1 show that the small-molecular-weight cyclic poly(γ-benzyl L-glutamate) was successfully formed.

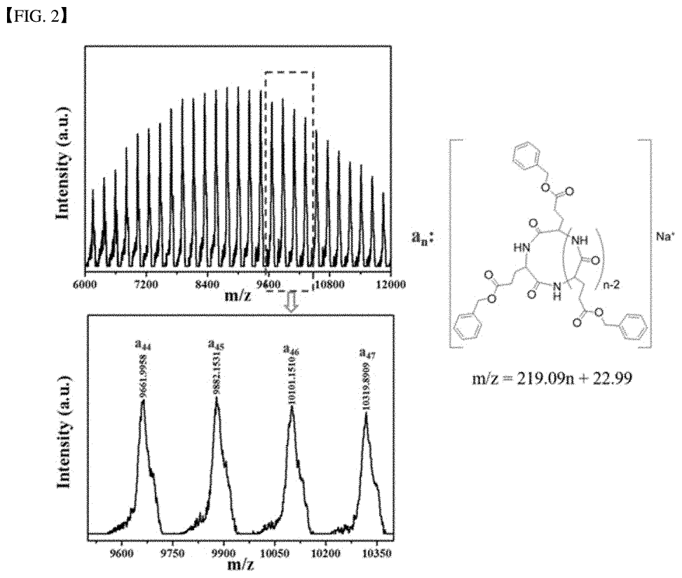

FIG. 2 shows a result of compound analysis. Specifically, FIG. 2 shows mass spectrometric analysis of poly(γ-benzyl L-glutamate) as produced according to the present disclosure. The mass spectrometry data was measured using DIT matrix (matrix-assisted laser desorption/ionization time-of-flight mass spectrometry (MALDI-TOF MS)). The mass spectrometry data in FIG. 2 show that poly(γ-benzyl L-glutamate) was successfully formed.

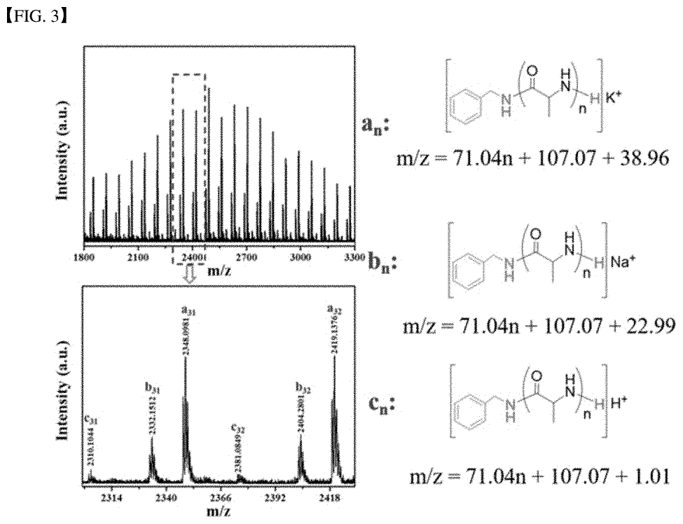

FIG. 3 shows a result of compound analysis. Specifically, FIG. 3 shows the mass spectrometric analysis of linear poly(L-alanine) as produced according to the present disclosure. The mass spectrometry data was measured using DIT matrix (matrix-assisted laser desorption/ionization time-of-flight mass spectrometry (MALDI-TOF MS)). As shown in FIG. 3, the mass spectrometry data confirmed the successful formation of linear poly(L-alanine).

ESI MS Measurement

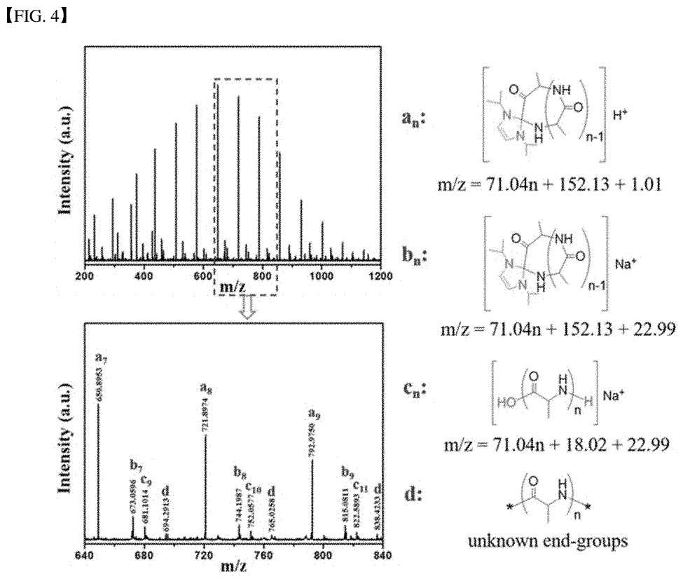

FIG. 4 shows a result of compound analysis. Specifically, FIG. 4 shows electrospray ionization mass spectrometry (ESI MS) of small-molecular-weight cyclic L-alanine as produced according to the present disclosure. As shown in FIG. 4, the mass spectrometry data confirmed that the cyclic compound was successfully formed.

FT-IR Measurement

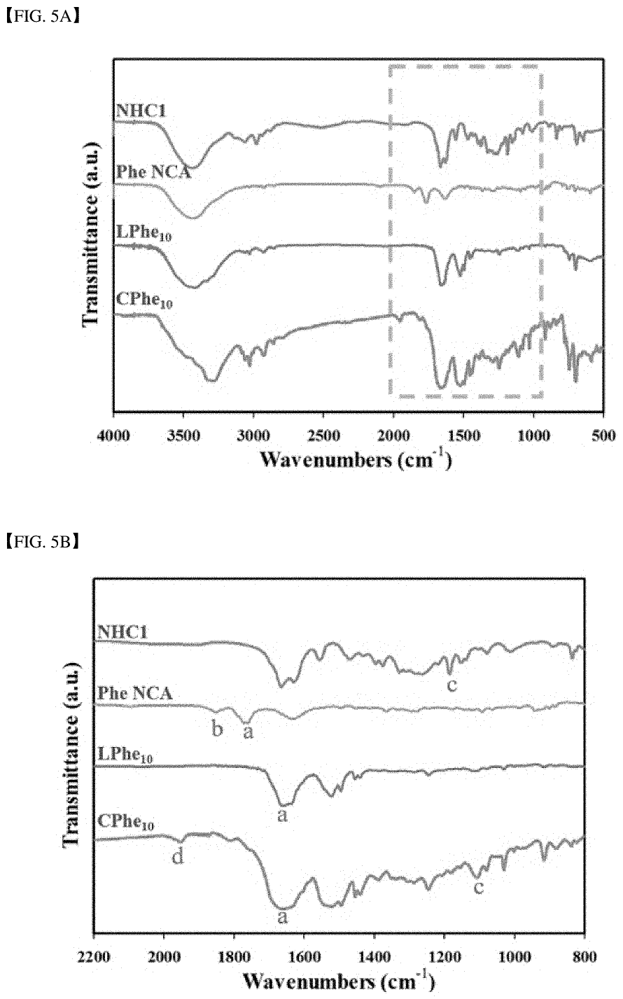

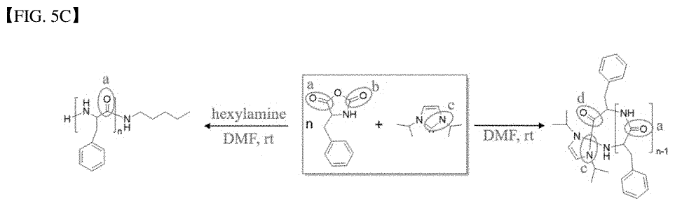

FIG. 5 shows a result of compound analysis. Specifically, FIG. 5 shows an analysis result using Fourier Transform Infrared Spectroscopy (FT-IR). The analysis results of N-heterocyclic carbene 1, phenylalanine N-carboxyanhydride, and compounds having a degree of polymerization of 10 as produced using the Examples of the present disclosure are shown in (A) of FIG. 5. For comparison, a portion of FIG. 5 (A) is enlarged and is shown as FIG. 5 (B). Referring to (B) and (C) in FIG. 5, 5-carbonyl forming CO₂ in the ring-opening polymerization of phenylalanine N-carboxyanhydride is indicated as “b”. 2-carbonyl representing a skeleton of each of linear/cyclic polyphenylalanine (LPhe10/CPhe10) is indicated as “a”. A stretching peak “d” (1953 cm⁻¹) corresponding to carbonyl adjacent to 1,3-diisoprephyl imidazolium in cyclic polyphenylalanine appears. An important peak “c” (1176 cm⁻¹) present in the N-heterocyclic carbene 1 exists in the cyclic polyphenylalanine. Thus, it was confirmed that the cyclic and linear peptides were successfully formed.

¹H, ¹H COSY Spectra Measurement

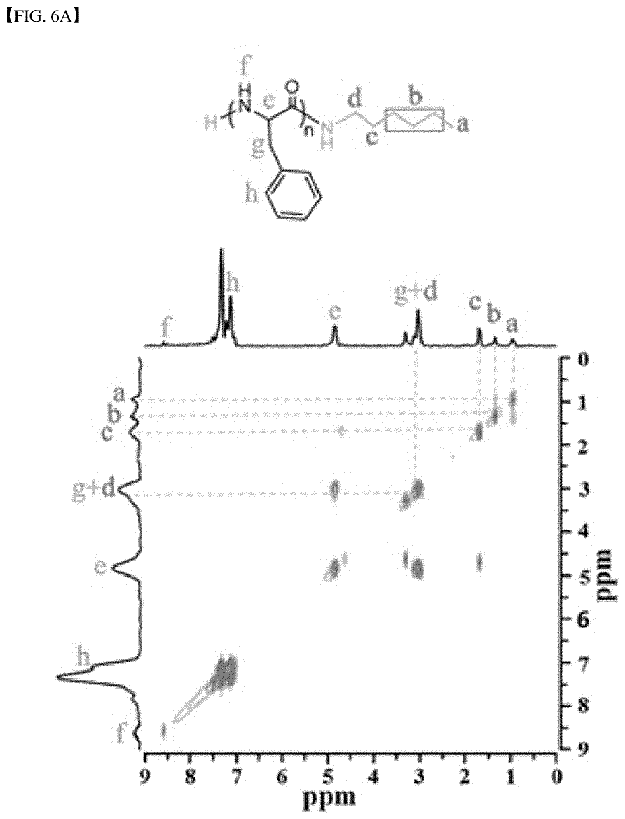

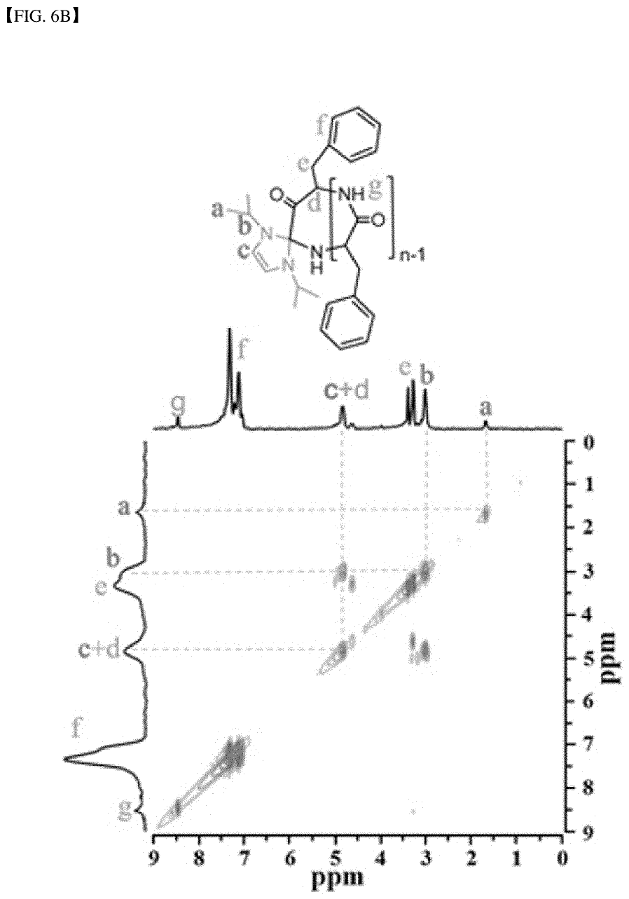

FIG. 6 shows a result of compound analysis. Specifically, FIG. 6 shows results of ¹H, ¹H correlation spectra (¹H, ¹H COSY spectra) analysis of phenylalanine compounds as produced using the Examples of the present disclosure. Referring to FIG. 6, (A) of FIG. 6 shows the analysis results for the linear L-phenylalanine produced according to the present disclosure, and FIG. 6 (B) shows the analysis results for the cyclic L-phenylalanine produced according to the present disclosure. When comparing a structure of each of the compounds (peak a to h) and the graph of the analysis results with each other, it may be confirmed that the cyclic and linear compounds were successfully formed.

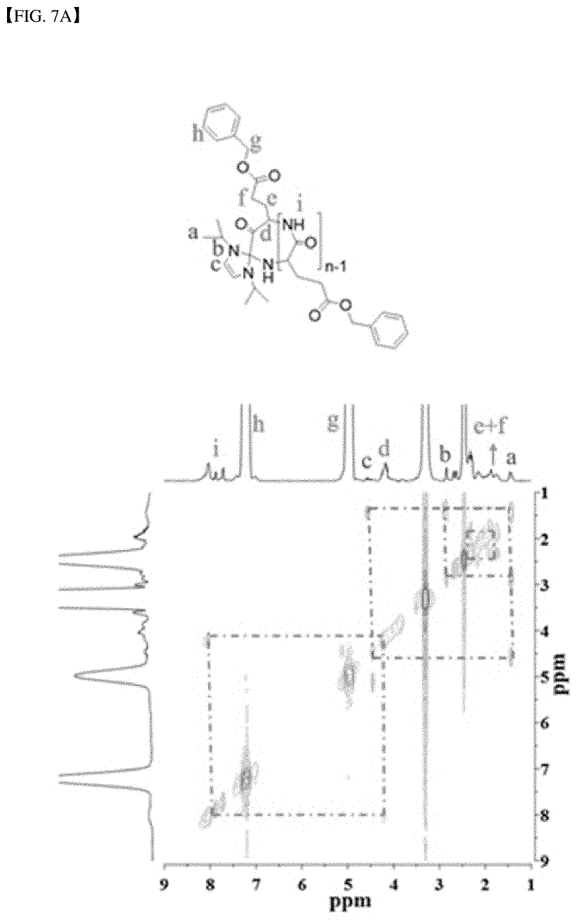

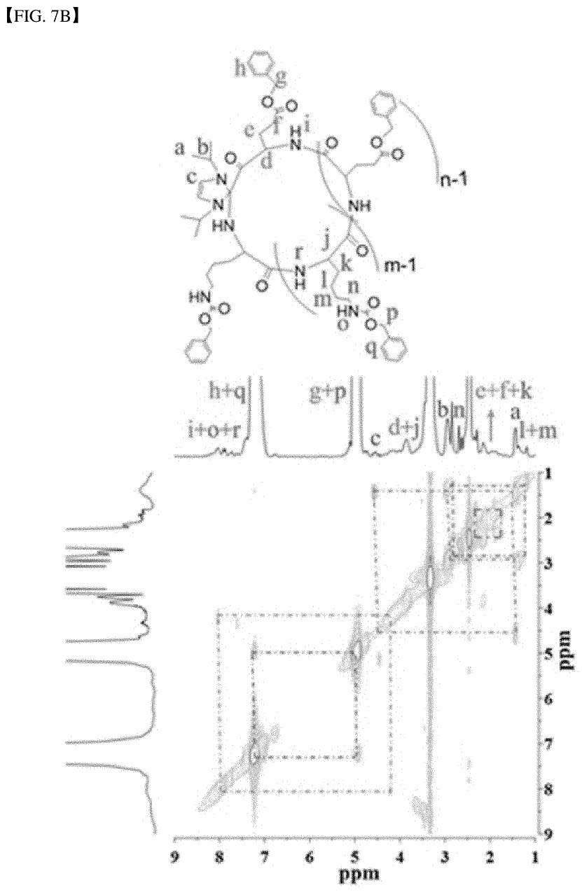

FIG. 7 shows a result of compound analysis. Specifically, FIG. 7 shows results of ¹H and ¹H correlation spectra (¹H, ¹H COZY spectra) analysis of poly(γ-benzyl L-glutamate) produced using the Examples of the present disclosure. Referring to FIG. 7, (A) of FIG. 7 shows the analysis results for the cyclic poly(γ-benzyl L-glutamate) produced according to the present disclosure, and (B) in FIG. 7 shows the analysis results for the block cyclic poly(γ-benzyl L-glutamate)-b-poly(ε-carbobenzoxy-L-lysine) produced according to the present disclosure. As shown in (A) in FIG. 7, there is a coupling relationship between peaks a/b and e/f in a range of about 1.5 to 2.8 ppm and, thus indicating that the cyclic poly(γ-benzyl L-glutamate) was formed. As shown in (B) in FIG. 7, new coupling relationships such as peaks 1+m/n, e+f+k, and d+j have been observed in a range of about 1.1 to 3.0 ppm, and a coupling relationship between the benzyl groups becomes stronger, thus indicating that the block cyclic poly(γ-benzyl L-glutamate)-b-poly(ε-carbobenzoxy-L-lysine) was formed.

Viscosity Measurement

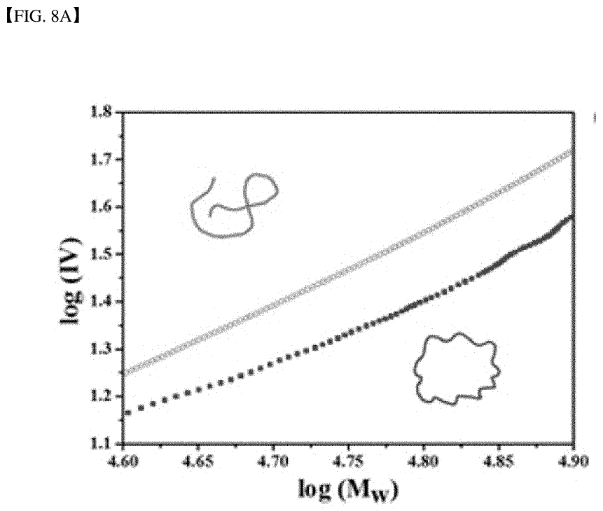

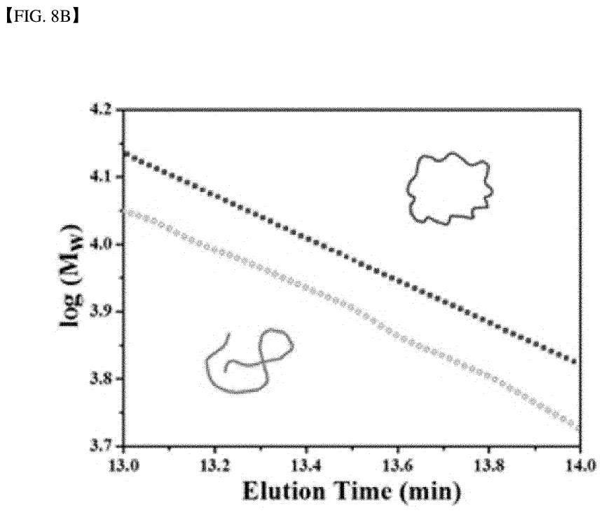

FIG. 8 shows a result of compound analysis. Specifically, FIG. 8 shows a graph of a Marj-Houwink equation and a relationship between a SEC elution time and an intrinsic viscosity of the compounds produced using the Examples of the present disclosure. (A) in FIG. 8 shows a graph of the Marj-Houwink equation of each of the cyclic poly(γ-benzyl L-glutamate) and the linear poly(γ-benzyl L-glutamate) as produced according to the Examples of the present disclosure. (B) in FIG. 8 (B) shows a graph showing the relationship between the SEC elution time and the intrinsic viscosity of each of the cyclic poly(γ-benzyl L-glutamate) and the linear poly(γ-benzyl L-glutamate) as produced according to the Examples of the present disclosure. As shown in (A) and (B) in FIG. 8, the compound having the cyclic structure has a lower intrinsic viscosity than the compound having the linear structure when the molecular weight is the same. Thus, the inherent viscosity of the cyclic poly(γ-benzyl L-glutamate) is lower than that of the linear poly(γ-benzyl L-glutamate), thus indicating that the cyclic poly(γ-benzyl L-glutamate) and the linear poly(γ-benzyl L-glutamate) were successfully formed.

SEC Measurement

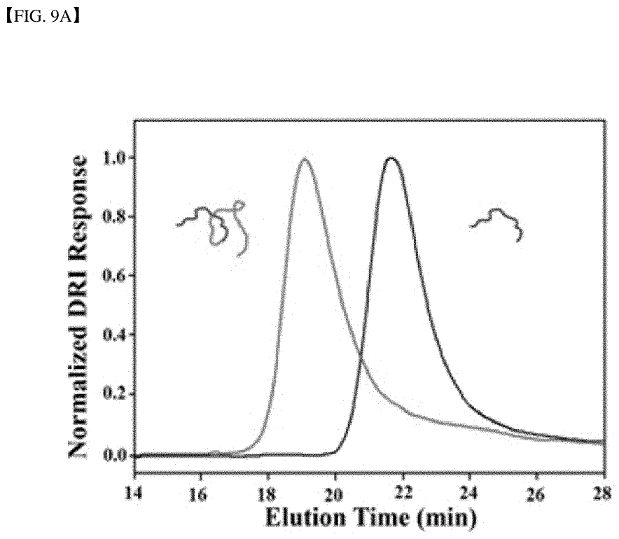

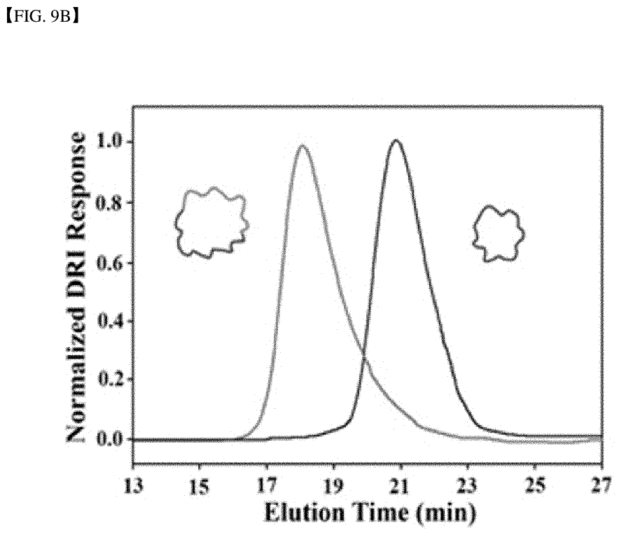

FIG. 9 shows a result of compound analysis. Specifically, FIG. 9 shows an analysis result of chromatography (SEC). (A) in FIG. 9 is a graph of analytical size exclusion chromatography (SEC) for block linear compounds as produced according to the Examples of the present disclosure. (B) in FIG. 9 is a graph of analytical size exclusion chromatography (SEC) for cyclic compounds as produced according to the Examples of the present disclosure. As shown in (A) in FIG. 9, a black line appearing later represents linear poly(γ-benzyl L-glutamate) ([M₁]₀/[I]₀=20/1). A red line appearing first represents linear poly(γ-benzyl L-glutamate)-b-poly(ε-carbobenzoxy-L-lysine) ([M₂]₀/[I]₀=60/1). This means that the block linear peptide is formed successfully. Further, as shown in (B) of FIG. 9, a black line appearing later represents cyclic poly(γ-benzyl L-glutamate) ([M₁]₀/[I]₀=30/1). A red line appearing first represents cyclic poly(γ-benzyl L-glutamate)-b-poly(ε-carbobenzoxy-L-lysine) ([M₂]₀/[I]₀=70/1). This means that the block cyclic peptide is formed successfully.

Although the disclosure has been described above with reference to the preferred Examples of the present disclosure, those skilled in the art will appreciate that various modifications and changes may be made in the present disclosure without departing from the spirit and scope of the present disclosure as set forth in the following claims.

What is claimed is:

1. A compound represented by a following Chemical Formula 1: 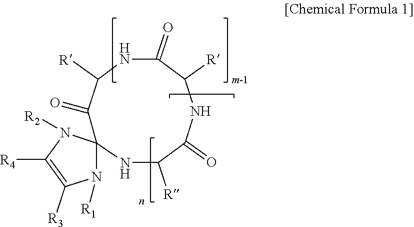
wherein in the Chemical Formula 1, each of R₁ to R₄ independently represents a hydrogen atom, an alkyl group having 1 to 20 carbon atoms, a cycloalkyl group having 3 to 20 carbon atoms, an ethylene glycol group having 3 to 50 carbon atoms, an aryl group having 6 to 20 carbon atoms, or a cycloalkenyl group having 5 to 20 carbon atoms,
wherein each of R′ and R″ independently represents R-A-(CH₂)ₓ—*, where A represents a single bond, a sulfur atom (—S—), an oxygen atom (—O—), a nitrogen atom (—N—), 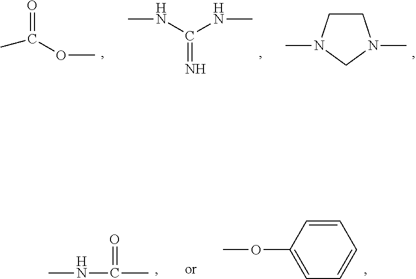
and R represents a hydrogen atom, a halogen atom, an alkyl group having 1 to 5 carbon atoms, an aryl group having 6 to 15 carbon atoms, a carbobenzoxy group, a trifluoroacetyl group, a carbonyl group, a triphenylmethyl group, a methoxydiphenylmethyl group, a 2,4,6-trimethoxybenzyl group, or a 2,2,4,6,7-pentamethyldihydrobenzofuran-5-sulfonyl group, where x represents an integer of 0 or greater,
wherein a hydrogen atom of each of R₁ to R₄, R′ and R″ may be independently substituted or unsubstituted with a substituent selected from a group consisting of a halogen atom, a sulfur atom, an oxygen atom, a hydroxy group, an amine group, an ether group, a carbonyl group, an alkenyl group, an allyl group, a phenyl group, and a cyano group,
wherein n is an integer greater than or equal to 0, and m is an integer of 1 or greater.

2. A method for producing a compound, the method comprising polymerizing α-amino acid N-carboxyanhydride using a catalyst represented by a following Chemical Formula 2: 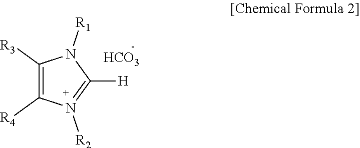
wherein in the Chemical Formula 2, each of R₁ to R₄ independently represents a hydrogen atom, an alkyl group having 1 to 20 carbon atoms, a cycloalkyl group having 3 to 20 carbon atoms, ethylene glycol having 3 to 50 carbon atoms, an aryl group having 6 to 20 carbon atoms or a cycloalkenyl group having 5 to 20 carbon atoms,
wherein a hydrogen atom of each of the alkyl group, the cycloalkyl group, the aryl group and the cycloalkenyl group may be independently substituted or unsubstituted with a substituent selected from a group consisting of an ether group, a carbonyl group, an alkenyl group, an allyl group, a halogen atom, a hydroxy group, a phenyl group, and a cyano group.

3. The method of claim 2, wherein the catalyst includes at least one of compounds represented by following Chemical Formulas 2-1, 2-2, 2-3 and 2-4:

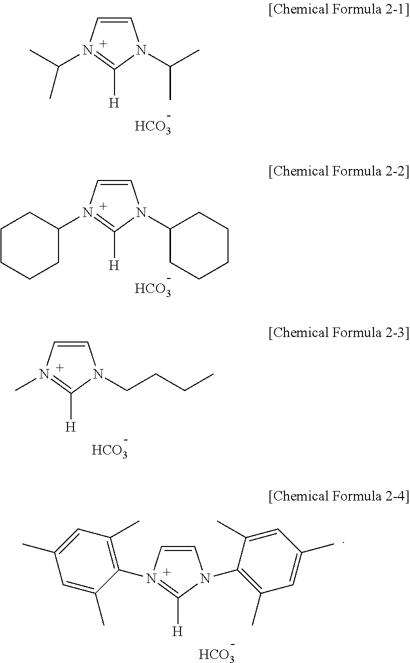

4. The method of claim 2, wherein the α-amino acid N-carboxyanhydride is represented by a following Chemical Formula 3: 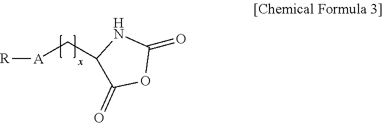
wherein in the Chemical Formula 3, A represents a single bond, a hydrogen atom (—H—), a sulfur atom (—S—), an oxygen atom (—O—), a nitrogen atom (—N—), 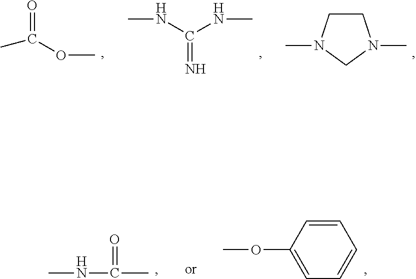
and R represents a hydrogen atom, a halogen atom, an alkyl group having 1 to 5 carbon atoms, an aryl group having 6 to 15 carbon atoms, a carbobenzoxy group, a trifluoroacetyl group, a carbonyl group, a triphenylmethyl group, a methoxydiphenylmethyl group, a 2,4,6-trimethoxybenzyl group, or a 2,2,4,6,7-pentamethyldihydrobenzofuran-5-sulfonyl group,
wherein x represents an integer of 0 or greater.

5. The method of claim 2, wherein the α-amino acid N-carboxyanhydride includes at least one selected from a group consisting of protected or unprotected L-glycine N-carboxyanhydride, L-alanine N-carboxyanhydride, L-phenylalanine N-carboxyanhydride, L-valine N-carboxyanhydride, L-luecine N-carboxyanhydride, L-methlonine N-carboxyanhydride, L-isoleucine N-carboxyanhydride, L-proline N-carboxyanhydride, L-tryptophan N-carboxyanhydride, L-serine N-carboxyanhydride, L-cysteine N-carboxyanhydride, L-aspartic acid N-carboxyanhydride, L-glutamate N-carboxyanhydride, L-lysine N-carboxyanhydride, L-arginine N-carboxyanhydride, L-histidine N-carboxyanhydride, L-asparagine N-carboxyanhydride, L-glutamine N-carboxyanhydride, L-threonine N-carboxyanhydride, and L-tyrosine N-carboxyanhydride.

6. The method of claim 2, wherein the α-amino acid N-carboxyanhydride may include at least one of compounds represented by following Chemical Formulas A, B, C, D, E, F, G, H, I, J and K: 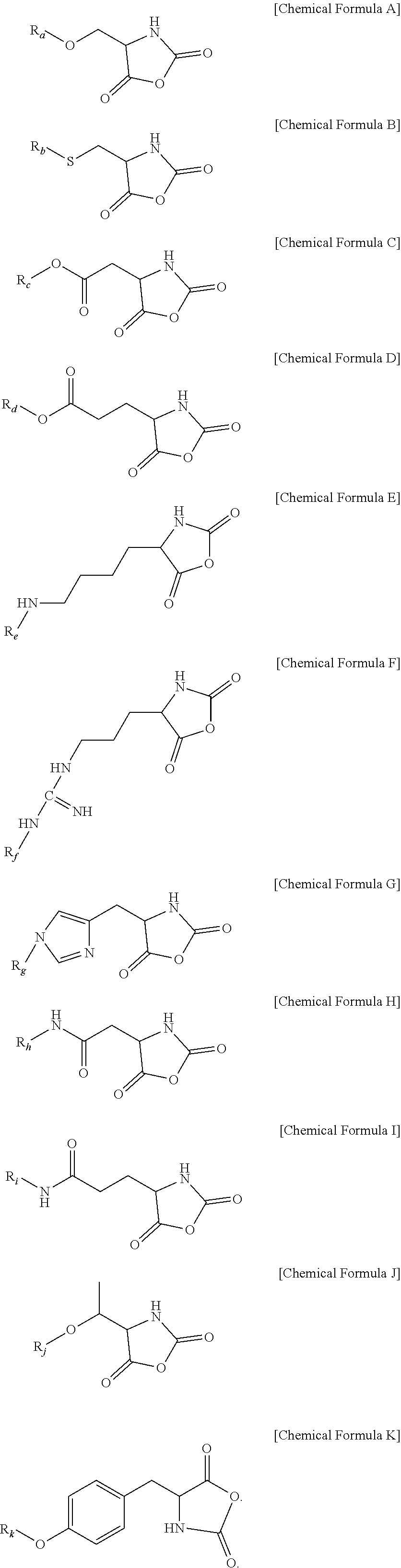
wherein each of Rₐ to Rₖ independently represents a hydrogen atom, a halogen atom, an alkyl group having 1 to 5 carbon atoms, an aryl group having 6 to 15 carbon atoms, a carbonyl group, a carbobenzoxy group, a trifluoroacetyl group, a triphenylmethyl group, a methoxydiphenylmethyl group, a 2,4,6-trimethoxybenzyl group, or a 2,2,4,6,7-pentamethyldihydrobenzofuran-5-sulfonyl group.

7. The method of claim 2, wherein the polymerization is performed in an inert gas atmosphere.

8. The method of claim 2, wherein the method further comprise, after the polymerization step, adding and reacting α-amino acid N-carboxyanhydride having the same structure as or a different structure from a structure of the α-amino acid N-carboxyanhydride used in the polymerization.

9. The method of claim 2, wherein the method produces the compound within 100 minutes.

10. The method of claim 2, wherein the compound produced using the method has a polydispersity index (PDI) of 1.5 or lower.

11. A compound containing:
a polymer ring structure formed using a compound represented by a following Chemical Formula 2 as a catalyst in a polymerization reaction of α-amino acid N-carboxyanhydride; and
imidazole of the compound represented by the following Chemical Formula 2, wherein the imidazole is bonded to the polymer ring structure while the imidazole shares a carbon atom constituting the polymer ring structure: 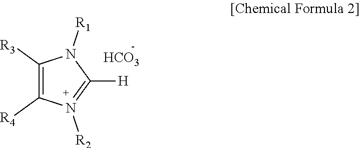
wherein in the Chemical Formula 2, each of R₁ to R₄ independently represents a hydrogen atom, an alkyl group having 1 to 20 carbon atoms, a cycloalkyl group having 3 to 20 carbon atoms, ethylene glycol having 3 to 50 carbon atoms, an aryl group having 6 to 20 carbon atoms or a cycloalkenyl group having 5 to 20 carbon atoms,
wherein a hydrogen atom of each of the alkyl group, the cycloalkyl group, the aryl group and the cycloalkenyl group may be independently substituted or unsubstituted with a substituent selected from a group consisting of an ether group, a carbonyl group, an alkenyl group, an allyl group, a halogen atom, a hydroxy group, a phenyl group, and a cyano group.

12. The compound of claim 11, wherein the catalyst includes at least one of compounds represented by following Chemical Formulas 2-1, 2-2, 2-3 and 2-4:

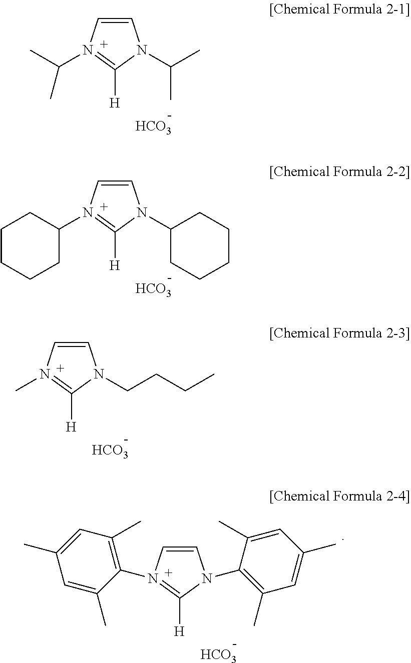

13. The compound of claim 11, wherein the α-amino acid N-carboxyanhydride is represented by a following Chemical Formula 3: 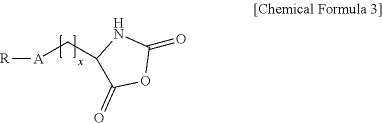
wherein in the Chemical Formula 3, A represents a single bond, a hydrogen atom (—H—), a sulfur atom (—S—), an oxygen atom (—O—), a nitrogen atom (—N—), 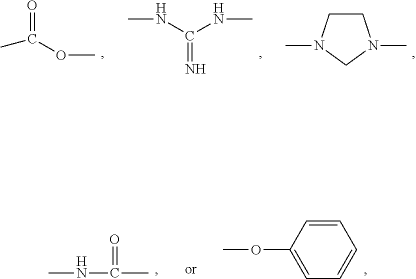
and R represents a hydrogen atom, a halogen atom, an alkyl group having 1 to 5 carbon atoms, an aryl group having 6 to 15 carbon atoms, a carbobenzoxy group, a trifluoroacetyl group, a carbonyl group, a triphenylmethyl group, a methoxydiphenylmethyl group, a 2,4,6-trimethoxybenzyl group, or a 2,2,4,6,7-pentamethyldihydrobenzofuran-5-sulfonyl group,
wherein x represents an integer of 0 or greater.

14. The compound of claim 11, wherein the α-amino acid N-carboxyanhydride includes at least one selected from a group consisting of protected or unprotected L-glycine N-carboxyanhydride, L-alanine N-carboxyanhydride, L-phenylalanine N-carboxyanhydride, L-valine N-carboxyanhydride, L-luecine N-carboxyanhydride, L-methlonine N-carboxyanhydride, L-isoleucine N-carboxyanhydride, L-proline N-carboxyanhydride, L-tryptophan N-carboxyanhydride, L-serine N-carboxyanhydride, L-cysteine N-carboxyanhydride, L-aspartic acid N-carboxyanhydride, L-glutamate N-carboxyanhydride, L-lysine N-carboxyanhydride, L-arginine N-carboxyanhydride, L-histidine N-carboxyanhydride, L-asparagine N-carboxyanhydride, L-glutamine N-carboxyanhydride, L-threonine N-carboxyanhydride, and L-tyrosine N-carboxyanhydride.

15. The compound of claim 11, wherein the α-amino acid N-carboxyanhydride includes at least one of compounds represented by following Chemical Formulas A, B, C, D, E, F, G, H, I, J and K: 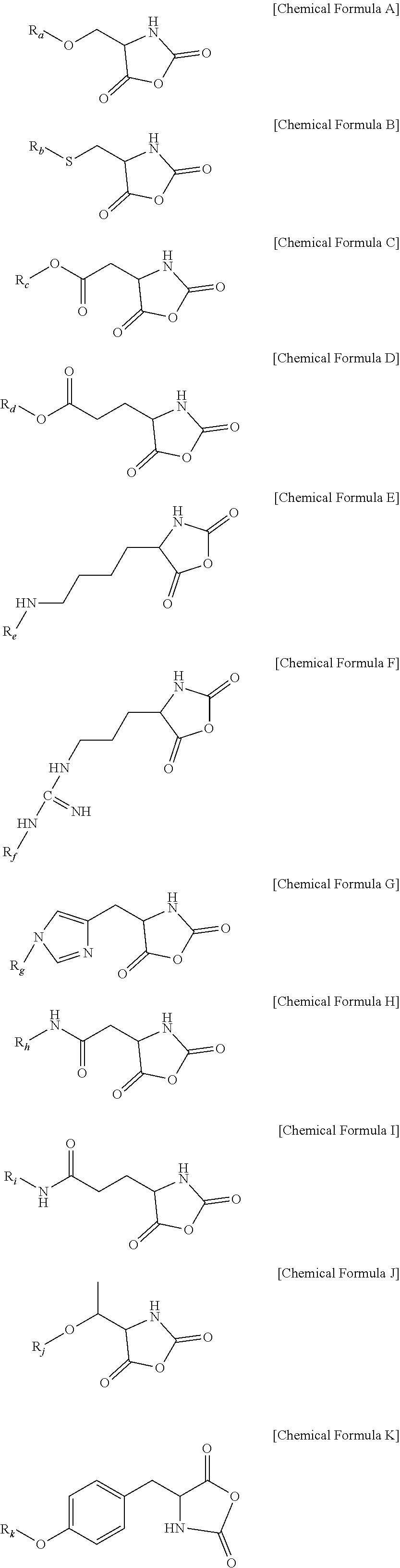
wherein each of Rₐ to Rₖ independently represents a hydrogen atom, a halogen atom, an alkyl group having 1 to 5 carbon atoms, an aryl group having 6 to 15 carbon atoms, a carbonyl group, a carbobenzoxy group, a trifluoroacetyl group, a triphenylmethyl group, a methoxydiphenylmethyl group, a 2,4,6-trimethoxybenzyl group, or a 2,2,4,6,7-pentamethyldihydrobenzofuran-5-sulfonyl group.
